(19) United States
(12) Patent Application Publication
(10) Pub. No.: US 2008/0206236 A1
(43) Pub. Date: Aug. 28, 2008

(54) Polyclonal antibody product

(75) Inventor: John Haurum, Charlottenlund (DK)

Correspondence Address: STERNE, KESSLER, GOLDSTEIN & FOX P.L.L.C., 1100 NEW YORK AVENUE, N.W., WASHINGTON, DC, 20005, US

(73) Assignee: Symphogen A/G, Lyngby (DK)

(21) Appl. No.: 12/068,654

(22) Filed: Feb. 8, 2008

Related U.S. Application Data
(60) Provisional application No. 60/900,325, filed on Feb. 9, 2007.

(30) Foreign Application Priority Data
Feb. 9, 2007 (DK) PA 2007 00222

(51) Int. Cl. A61K 39/00; C12N 5/00; G01N 33/00

(52) U.S. Cl. 424/130.1; 435/326; 435/7.1

(57) ABSTRACT

The present invention relates to a composition comprising two or more sets of target-specific recombinant polyclonal antibodies, each set being capable of binding to a distinct target, wherein said distinct targets are related to diseases, which may be treated or prevented by the use of antibodies, and wherein said composition is essentially free from immunoglobulin molecules that do not bind to one of said distinct targets. Further the application describes pharmaceutical and diagnostic compositions comprising said composition and use in prevention, treatment and/or amelioration of one or more diseases.

This application claims the benefit of U.S. Appl. No. 60/900,325, filed Feb. 9, 2007, and Denmark Pat. Appl. No. PA 2007 00222, filed Feb. 9, 2007, both of which are hereby incorporated by reference in their entirety.

### BACKGROUND OF THE INVENTION

1. Field of the Invention

The present invention relates to a composition comprising two or more sets of target-specific recombinant polyclonal antibodies, each set being capable of binding to a distinct target, wherein said distinct targets are related to diseases, which may be treated or prevented by the use of antibodies, and wherein said composition is essentially free from immunoglobulin molecules that do not bind to one of said distinct targets. Further the application describes pharmaceutical and diagnostic compositions comprising said composition and use in prevention, treatment and amelioration of one or more diseases.

2. Background Art

Antibodies are a central factor in the immunity against invading pathogens such as bacteria and viruses, as well as against malignantly transformed cells. The natural antibody response is polyclonal, comprising antibodies against several antigens and epitopes, thus increasing the probability of eliminating the invading pathogen or malignant cell. The pharmacological advantages of polyclonality have been exploited in the use of plasma-derived immunoglobulin products to treat a number of infectious diseases. However, the use of plasma-derived products is limited by their cost, inconvenience of use, limited supply, and potential for transferring diseases from the donor to the patient.

The key feature of antibodies is their ability to bind with high specificity and affinity to complex soluble or membrane-bound structures on disease-associated targets. The specificity of the individual antibody molecule is determined by so-called variable regions encoded by a diverse set of genes, which in combination with splicing events and mutations give rise to as many as 10¹⁰ different antibody specificities. The most basic antibody function is neutralization, merely mediated by antibodies binding to their target and inhibiting its binding to, for example, a receptor. Antibodies can also mediate clearance of soluble or cellular targets, either by formation of so-called immune complexes that are removed in the kidney, or through phagocytosis. Finally, antibodies can eliminate cellular targets by recruiting cytotoxic systems, such as complement or cytotoxic cells.

As therapeutic molecules, antibodies have a number of favorable attributes; their effect is immediate, they have a relatively long in vivo half-life, and, apart from on-target effects, antibodies are generally considered non-toxic. The use of therapeutic antibodies has been known for more than a century. Pioneered by Kitasato and von Behring, so-called passive immunotherapy was initially based on the transfer of animal serum to humans infected with cowpox virus and measles. The use of animal serum was limited by side effects. Development of improved plasma fractionation technologies and the availability of products based on human plasma circumvented many of the side effects and increased the clinical applicability of plasma-derived antibody products.

More than 30 years ago, Köhler and Milstein invented the technology to produce monoclonal antibodies (mAbs) by immortalizing murine B cells. This initiated a whole new era leading to a revolution in the pharmaceutical industry, especially with the development of chimeric, humanized and fully human mAbs with better safety and pharmacodynamic profiles than the murine mAbs. However, it is becoming increasingly clear that although mAbs is promising for treatment of a variety of indications, successful treatment or prophylaxis of complex diseases such as infections require multivalent medical products.

In WO 2004/010935 it has been suggested to combine monoclonal antibodies for the treatment and prevention of the viral respiratory infections caused by respiratory syncytial virus, human metapneumovirus, and parainfluenza virus.

Cloning and manufacturing of a recombinant polyclonal antibody have been described in two patent applications (WO 2005/042774, and WO 2004/061104, respectively). The techniques described in these two patent applications allow identification and industrial manufacturing of a recombinant human polyclonal antibody for single medical use in humans. However, in more complex situations where more than one disease may be encountered, a single recombinant polyclonal antibody as mentioned above may not be sufficient.

### SUMMARY OF THE INVENTION

The present invention provides a composition comprising two or more sets of target-specific recombinant polyclonal antibodies, each set being capable of binding to a distinct target, wherein said distinct targets are related to diseases, which may be treated or prevented by the use of antibodies, and wherein said composition is essentially free from immunoglobulin molecules that do not bind to one of said distinct targets. As opposed to a plama derived immunoglobulin preparation the recombinant nature of the antibody compositions of the present invention makes it possible to manufacture an antibody composition with known specificity and in a safe manner. Therefore the antibody compositions of the present invention comprise the antibodies intended for a particular use, and no further antibodies.

The application also describes pharmaceutical and diagnostic compositions comprising said composition and its use in prevention, treatment and amelioration of one or more diseases.

The invention furthermore provides a method for generating in a single batch a composition comprising two or more sets of target-specific recombinant polyclonal antibodies, each set being capable of binding to a distinct target, wherein said distinct targets are related to diseases, which may be treated or prevented by the use of antibodies, and wherein said composition is essentially free from immunoglobulin molecules that do not bind to one of said distinct targets. Said method allows not only for the control of the binding specificity of the antibodies produced but also for the control of the effector-function of the antibodies.

The present invention provides a composition comprising two or more sets of target-specific recombinant polyclonal antibodies (in short a polycomposition), each set of target-specific recombinant polyclonal antibodies being capable of binding to a distinct target, wherein said distinct targets are related to diseases, which may be treated or prevented by the use of antibodies. The composition is essentially free from immunoglobulin molecules that do not bind to one of said distinct targets.

The present polycomposition may be used in pharmaceutical compositions for therapy and/or prophylaxis of an individual who is suffering from two or more diseases which are likely to occur at the same time and/or in the same organ system so that simultaneous administration of the two or more sets of target-specific recombinant polyclonal antibodies (in short “a polycomposition”) improves the therapeutic regimen.

The present polycomposition may also be used in pharmaceutical compositions for therapy and prophylaxis of an individual who is considered to be at risk of suffering from one or more diseases which are likely to occur at the same time and/or in the same organ system so that a simultaneous administration of the polycomposition improves or the prophylaxis or the therapeutic regimen or treats one or more symptoms of the one or more diseases. The polycomposition may also improve the likelihood of efficacy in the treatment or prevention.

Thus, the present polycomposition may be used for treatment or prophylaxis of an individual potentially suffering from different types of infectious diseases, allergy, asthma and/or other respiratory diseases, among other diseases. In the context of the present invention, “one or more diseases” may include two or more diseases, such as three or more diseases, for example four or more diseases, such as five or more diseases, or even higher number of diseases. Different diseases may show the same clinical symptoms such as infections caused by different vira or different bacteria.

Further, the present polycomposition may be used in pharmaceutical compositions for therapy and prophylaxis of an individual who has been or who is considered to be in risk of having been or becoming exposed to harmful or potentially harmful agents in a particular environmental niche.

Further, the present polycomposition may be used in pharmaceutical compositions for therapy and prophylaxis of an individual who has been or who is considered to be in risk of having been or becoming exposed to harmful or potentially harmful agents in a particular environmental niche wherein the agents have not been identified in every detail, but for which sufficient information is available to conclude that the agents potentially are present and harmful and that a treatment or prophylaxis with antibodies directed against targets of said agents may be effective.

Further, the present polycomposition may be used in pharmaceutical compositions for therapy or prophylaxis of an individual in risk of suffering of one or more secondary infections commonly associated with a primary disease and for which secondary disease prophylaxis or therapy may be desirable.

The particular environment niche may be a hospital environment, a food-related environment, a water-related environment, sources of allergens, a potential or actual site of biological warfare or bioterrorism, or a site with potential or actual exposure to pathogenic, toxic, potentially pathogenic, or potentially toxic agent(s).

The present invention also provides a method for generating a polycomposition according to the invention, whereby two or more polyclonal cell lines each containing a number of different antibody coding sequences capable of directing the expression of the resulting antibodies, are combined in a single container and cultured under conditions facilitating expression of the antibodies and recovering the antibodies from the cell culture cells or cell culture supernatant. The method according to the invention allows not only for the control of the binding specificity of the antibodies but also for the control of the effector function of the antibodies in that this is controlled by the constant region of the antibody. The sequence encoding the constant region of an antibody is controllable in the present invention e.g. by selecting a particular constant region sequence to be present in the vector to which the sequences encoding the variable sequences of the antibody are transferred (the recipient vector) thereby creating the expression vector. Use of different recipient vectors harboring particular antibody constant regions provides a very easy way of controlling the effector function of the composition according to the invention. Using the same constant region for all antibodies in a composition of the invention eases down stream processing and characterization considerably.

### DEFINITIONS

The term “antibody” describes a functional component of serum and is often referred to either as a collection of molecules (antibodies or immunoglobulins, fragments, etc) or as one molecule (the antibody molecule or immunoglobulin molecule). An antibody molecule is capable of binding to or reacting with a specific antigenic determinant (the antigen or the antigenic epitope), which in turn may lead to induction of immunological effector mechanisms. An individual antibody molecule is usually regarded as monospecific, and a composition of antibody molecules may be monoclonal (i.e., consisting of identical antibody molecules) or polyclonal (i.e., consisting of different antibody molecules reacting with the same or different epitopes on the same antigen or on distinct, different antigens). The distinct and different antibody molecules constituting a polyclonal antibody may be termed “members”. Each antibody molecule has a unique structure that enables it to bind specifically to its corresponding antigen, and all natural antibody molecules have the same overall basic structure of two identical light chains and two identical heavy chains. Antibodies are also known collectively as immunoglobulins. The terms antibody or antibodies as used herein are used in the broadest sense and cover intact antibodies, chimeric, humanized, fully human and single chain antibodies, as well as binding fragments of antibodies, such as Fab, Fv fragments or scFv fragments, as well as multimeric forms such as dimeric IgA molecules or pentavalent IgM.

The term “polyclonal antibody” describes a composition of different (diverse) antibody molecules which are capable of binding to or reacting with several different specific antigenic determinants on the same or on different antigens. Usually, the variability of a polyclonal antibody is located in the so-called variable regions of the polyclonal antibody, in particular in the CDR regions. In the present invention a mixture of two or more polyclonal antibodies (a polycomposition) is preferably produced in one pot (a single container) from a polyclonal polycomposition cell line, which is produced from two or more parental polyclonal cell lines each expressing antibody molecules which are capable of binding to a distinct target, but it may also be a mixture of two or more polyclonal antibodies produced separately. A mixture of monoclonal antibodies providing the same antigen/epitope coverage as a polyclonal antibody of the present invention will be considered as an equivalent of a polyclonal antibody. When stating that a member of a polyclonal antibody binds to an antigen, it is herein meant to be binding with a binding constant below 100 nM, preferably below 10 nM, even more preferred below 1 nM.

The term “recombinant antibody” is used to describe an antibody molecule or several molecules that is/are expressed from a cell or cell line transfected with an expression vector comprising the coding sequence of the antibody which is not naturally associated with the cell. If the “antibody molecules” in a “recombinant antibody” are diverse or different, the term “recombinant polyclonal antibody” applies in accordance with the definition of a “polyclonal antibody”.

The term “a composition comprising two or more sets of target-specific recombinant polyclonal antibodies”, in short called a polycomposition”, describes a composition comprising antibody members of two or more target-specific recombinant polyclonal antibody sets, wherein the antibody members of each set of target-specific recombinant polyclonal antibodies are capable of binding to a distinct target.

The term “the composition is essentially free from immunoglobulin molecules that do not bind to one of the distinct targets” means that more than 80% of the antibodies in the composition, preferably more than 90%, more preferably more than 95% and most preferably more than 99%, bind to or are capable of binding to one of the distinct targets.

The term “immunoglobulin” commonly is used as a collective designation of the mixture of antibodies found in blood or serum, but may also be used to designate a mixture of antibodies derived from other sources, or may be used synonymous for the term “antibody”.

The classes of human antibody molecules are: IgA, IgD, IgE, IgG and IgM. Members of each class are said to be of the same isotype. IgA and IgG isotypes are further subdivided into subtypes. The subtype(s) of IgA and IgG commonly refers to IgA₁, IgA₂ and IgG₁, IgG₂, IgG₃ and IgG₄, respectively.

The terms “cognate V_{H} and V_{L} coding pair” or “cognate pairs of V_{H} and V_{L} sequences” describes an original pair of V_{H} and V_{L} coding sequences contained within or derived from the same cell. Thus, a cognate V_{H} and V_{L} pair represents the V_{H} and V_{L} pairing originally present in the donor from which such a cell is derived. The term “an antibody expressed from a V_{H} and V_{L} coding pair” indicates that an antibody or an antibody fragment is produced from a vector, plasmid or similar containing the V_{H} and V_{L} coding sequences. When a cognate V_{H} and V_{L} coding pair is expressed, either as a complete antibody or as a stable fragment thereof, they preserve the binding affinity and specificity of the antibody originally expressed from the cell they are derived from. A composition of cognate pairs is also termed a repertoire of cognate pairs, and may be kept individually or pooled.

The term “epitope” is commonly used to describe a site on an antigen to which the antibody will bind. An antigen is a substance that stimulates an immune response, e.g. toxin, virus, bacteria, proteins or DNA. An antigen often has more than one epitope, unless they are very small. Antibodies binding to different epitopes on the same antigen can have varying effects on the activity of the antigen they bind depending on the location of the epitope. An antibody binding to an epitope in an active site of the antigen may block the function of the antigen completely, whereas another antibody binding at a different epitope may have no or little effect on the activity of the antigen. Such antibodies, may however still activate complement or other effector mechanisms and thereby result in the elimination of the antigen.

The term “a distinct target” is used to denote an antigen or a group of antigens associated with a particular disease which may be treated or prevented by the use of antibodies.

The term “distinct targets originating from a particular environmental niche” refers to antibody targets on pathogens or toxic agents, which may be frequently occurring in a particular environmental niche, e.g. a hospital, a factory setting, a public place, a restaurant, a nursery home, child care institution, a farm, an element of a transport system such as a train, bus, station, subway, ship, or plane, etc. Many different types of places or environmental niches may constitute an increased risk of exposure to certain pathogens or toxic agents, which contain targets that may be addressed with a polyclonal antibody.

The term “a particular environmental niche” denotes an environment where two or more distinct potentially harmful targets co-exist or potentially co-exist, so that when an individual is exposed to at least one of said targets, sufficient information is available about the environmental niche to conclude that a treatment or prophylaxis based on a polycomposition according to the invention comprising two or more sets of recombinant polyclonal antibodies specifically binding to the distinct targets expected to be present in the particular environmental niche would be beneficial for the individual even in the absence of a diagnosis.

The term “nosocomial infections” denotes infections acquired in hospitals and other institutions taking care of people not being able to take care of themselves, e.g. elderly homes and daycare centers.

The term “fully human” used for example in relation to DNA, RNA or protein sequences describes sequences which are between 98 to 100% homologous with human sequences.

The term “mirrors the humoral immune response” when used in relation to a polyclonal antibody refers to an antibody composition where the nucleic acid sequences encoding the individual antibody members are derived from a donor in such a way that the variable heavy chain (V_{H}) and the variable light chain (V_{L}) are maintained in the pairs as originally present in the donor (cognate pairs). In order to mirror the diversity of a humoral immune response in a donor, all the nucleic acid sequences encoding antibodies which bind to relevant antigens are selected based on a screening procedure. The isolated sequences are analyzed with respect to diversity of the variable regions, in particular the CDR regions, but also with respect to the V_{H} and V_{L} family. Based on these analyses a population of cognate pairs representing the overall diversity of the antibodies binding to relevant antigens is selected.

A polyclonal antibody typically has at least 2, 3, 4, 5, 6, 8, 10, 20, 30, 40, 50, 100, 1000 or 10⁴ distinct members.

The term “therapy” refers to treatment of a disease or condition. Treatment is not necessarily curative, and may also be ameliorative.

The term “prophylaxis” or “prevention” refers to administration of medicine for the prevention of a disease or condition.

A composition is said to be “pharmacologically acceptable” if its administration can be tolerated by a recipient patient without undue side-effects.

The term “a unit dose form” denotes a “ready-to-administer” form comprising a therapeutically effective amount of the active ingredient.

The term “state of immunodeficiency” refers to either inherited or acquired disorders in which some aspect or aspects of the individual's immune response are absent or functionally defective. Such disorders include congenital immunodeficiency, Acquired Immunodeficiency Syndrome (AIDS) caused by HIV infection, or immunodeficiency secondary to malignant disease, e.g. hematological malignancy-induced bone-marrow suppression, etc.

The term “immune response” refers to the molecules, cells, organs and processes involved in the host immune defense.

The term “state of immunosuppression” refers to a condition where an individual's immune response is inhibited by exogenous agents, e.g. medicines that inhibit immune responses such as those used in the treatment or prevention of graft rejection and severe autoimmune disease. The term also refers to cases where an individual receives pharmacological or radiological treatments that prevent cell proliferation or cause cell death, such as in the therapy of cancer. Immunosuppression is also a common consequence of major injury, including surgery, splenectomy, trauma, shock, burns, infection or sepsis.

The terms “state of immunosuppression” and “state of immunodeficiency” may be used interchangeably.

The term “recombinant polyclonal cell line” or “polyclonal cell line” refers to a mixture/population of protein-expressing cells that are transfected or transduced with a repertoire of variant nucleic acid sequences (e.g. a repertoire of antibody-encoding nucleic acid sequences), such that the individual cells, which together constitute the recombinant polyclonal cell line, each carry at least one transcriptionally active copy of a distinct nucleic acid sequence of interest, which encodes one member of the recombinant polyclonal antibody of interest. In one embodiment of the invention, only a single copy of a distinct nucleic acid sequence is integrated at a specific site in the genome of each of the cells. The cells constituting the recombinant polyclonal cell line are selected for their ability to retain the integrated copy of the distinct nucleic acid sequence of interest, for example by antibiotic selection. Cells which can constitute such a polyclonal cell line can be for example bacteria, fungi, eukaryotic cells, such as yeast, insect cells, plant cells or mammalian cells, especially immortal mammalian cell lines such as CHO cells, COS cells, BHK cells, myeloma cells (e.g., Sp2/0 cells, NS0), NIH 3T3, YB2/0 and immortalized human cells, such as HeLa cells, HEK 293 cells, or PER.C6.

The terms “pMCB” and “pWCB” denote a polyclonal master cell bank and a polyclonal working cell bank, respectively, i.e. a cell bank comprising cell lines expressing members of a recombinant polyclonal antibody.

The term “potentially occurring” is used to describe situations where knowledge about the personal or medical history of the individual provides knowledge of potential co-existence of one or more secondary diseases, or knowledge about a certain likely exposure to one or more potentially pathogenic or noxious substances makes it likely above average in the population that a person or patient harbors one or more targets which may be treated by administration of polyclonal antibodies. Sometimes the likelihood cannot be estimated numerically but the suspicion that a particular exposure has taken place is raised for other reasons, e.g. fear of contamination or infection due to an accident, attack, animal bite etc.

The terms “mixed pMCB” and “mixed pWCB” denote a mixed polyclonal master cell bank and a mixed polyclonal working cell bank, respectively, i.e. a cell bank comprising cell lines expressing members of a polycomposition according to the invention.

The terms “sequences encoding V_{H} and V_{L} pairs” or “V_{H} and V_{L} encoding sequence pairs” indicate nucleic acid molecules, where each molecule comprise a sequence that code for the expression of a variable heavy chain and a variable light chain, such that these can be expressed as a pair from the nucleic acid molecule if suitable promoter and/or IRES regions are present and operably linked to the sequences. The nucleic acid molecule may also code for part of the constant regions or the complete constant region of the heavy chain and/or the light chain, allowing for the expression of a Fab fragment, a full-length antibody or other antibody fragments if suitable promoter and/or IRES regions are present and operably linked to the sequences.

A pharmaceutical composition comprising the present polycomposition is said to be administered in a “therapeutically effective amount” if the amount administered is physiologically significant, e.g. prevents or attenuates the effect of at least one distinct target.

The following abbreviations are used: GVHD=graft versus host disease; HIV=Human Immunodeficiency virus; pWCB=polyclonal working cell bank; pMCB=polyclonal master cell bank; vv-rpAB=Vaccinia virus recombinant polyclonal antibody; RhD-rpAb=RhesusD recombinant polyclonal antibody.

### DESCRIPTION OF THE DRAWINGS

FIG. 1: shows the cell viability of the seed trains in the present experiment. One frozen ampoule of Sym001 pWCB and one ampoule of Sym002 pWCB were thawed separately in T-flasks on day 0 (D0) to recover. The next day (D₁) each of the two cultures were transferred to separate shaker flasks for cell propagation and initiation of seed trains for inoculation of bioreactors. On day 4 (D₄) a mixture of the Sym001 pWCB and Sym002 pWCB cells (cell mixture 1:1) was prepared in a shaker flask and cultivated until inoculation on day 10 (D₁₀) of a bioreactor (experiment FCW 088). In parallel, the Sym001 pWCB and the Sym002 pWCB cells were kept in separate shaker flasks during the seed train, until day 10 (D₁₀), where a mixture (cell number 1:1) was prepared and inoculated into a bioreactor (experiment FCW089). Cell number and viability were determined using a Vi-CELL™-cell viability analyzer.

FIG. 2: shows the viable cell number, the viability and the IgG production in units in the bioreactor FCW 088. On day 10 (D₁₀) the bioreactor FCW 088 was inoculated with 1.34×10⁶ cell/ml from the seed train consisting of Sym001+Sym002 pWCB cells mixed at day 4. The cell culture was inoculated in 3 L medium. At 46 hours post inoculation 2 L medium was supplemented to the two bioreactors (FCW 088 and FCW 089, see FIG. 3). A temperature downshift was performed at 97 hours after inoculation. The bioreactor was cultivated in batch mode and the cell culture supernatant was harvested, when cell viability was ˜55%, at 137 hours after inoculation of the bioreactor.

FIG. 3: shows the viable cell number, the viability and the IgG production in units in the bioreactor FCW 089. On day 10 (D₁₀) the bioreactor FCW 089 was inoculated with a mix of cells from the two separate Sym001 and Sym002 seed trains. The cells were mixed with equal cell numbers (1:1) and a total amount of 1.33×10⁶ mixed cells/ml were used for the inoculation and suspended in 3 L medium. At 46 hours post inoculation 2 L medium was supplemented to the two bioreactors. A temperature downshift was performed at 97 hours after inoculation. The bioreactor was cultivated in batch mode and the cell culture supernatant was harvested, when cell viability was ˜55%, at 137 hours after inoculation of the bioreactor.

FIG. 4: shows a comparison between bioreactor FCW 088 and FCW 089 with respect to viable cell number and IgG production during the bioreactor production runs from day 10 (D₁₀) until harvest at day 16 (D₁₆) after thawing of Sym001 pWCB and Sym002 pWCB.

FIG. 5 illustrates the cation exchange chromatographic (IEX) profiles of Sym001 working standard; Sym002 working standard; a mix (1:1) of Sym001 working standard and Sym002 working standard after purification of the supernatants by IgG capture on protein-A columns using conventional antibody purification techniques. Briefly, cation-exchange chromatography was performed employing a PolyCAT A column (4.6×100 mm, from PolyLC Inc., MD) in 25 mM sodium acetate, 150 mM sodium chloride, pH 5.0 at a flow rate of 60 ml/h at room temperature. Elution of the antibody peaks was subsequently done using a linear gradient from 150 mM to 350 or 500 mM NaCl in 25 mM sodium acetate, pH 5.0. The antibody peaks were detected by spectrophotometry at 280 nm. The PolyCAT A column separates individual antibody members from a polyclonal antibody composition based on differences in net charge between individual antibodies.

FIG. 6: illustrates the IEX profile of FCW 088 after purification of the supernatants by IgG capture on protein-A columns using conventional antibody purification techniques as described above. The IEX profile of FCW 088 is compared with IEX chromatograms of the SYM001 and the SYM002 working standards.

FIG. 7: illustrates the IEX profile of FCW 089 after purification of the supernatants by IgG capture on protein-A columns using conventional antibody purification techniques as described above. The IEX profile of FCW 089 is compared with IEX chromatograms of the SYM001 and the SYM002 working standards.

FIG. 8: shows a comparison of the IEX chromatograms of FCW 088 and FCW 089.

FIG. 9: illustrates an overlay of the IEX profiles from FCW 088 (black) and FCW 089 (gray). The profiles are very similar and indicate that the co-production of Sym001 and Sym002 is reproducible.

FIGS. 10 A-F: show binding of the polycomposition comprising anti-vv rpAb and anti-RhD rpAb antibodies to two different vaccinia virus strains as well as to 4 different vaccinia virus antigens as measured by Elisa after coating of the vaccinia virus antigens. Each figure illustrates a comparison of the recombinant polyclonal antibody poly-compositions from FCW 088 and FCW 089 with a Sym002 working standard (positive control), a Sym001 working standard (negative control), a Sym001/Sym002 working standard mixture (1:1) and a blank. A) The IHD-W vaccinia virus sub-strain (5 μg/ml) was used for coating of the Elisa plate; B) The B5R vaccinia virus antigen (1 μg/ml) used as coating; C) The Lister strain (5 μg/ml) used as coating; D) The A33 (1 μg/ml) vaccinia virus antigen used as coating; E) The VCP (1 μg/ml) vaccinia virus antigen used as coating and F) the A27L (1 μg/ml) vaccinia virus antigen used as coating. For each experiment the samples in 6 dilutions ranging from 0.5 μg/ml to 0.0001 μg/ml were analyzed on the listed coatings in duplicate.

### DETAILED DESCRIPTION OF THE INVENTION

### Targets and Polycompositions

A polycomposition according to the present invention is composed of a number of antibody molecules. Each molecule in the polycomposition is included based on its ability to bind an epitope/antigen which is associated with a distinct target and which together with other molecules binding to the same target constitute a target-specific recombinant polyclonal antibody set. The polycomposition comprises two or more such sets of target-specific recombinant polyclonal antibodies, wherein the distinct targets are different disease-causing agents, e.g. pathogens which may cause disease that can be treated or prevented by use of antibodies.

The polycomposition is particularly advantageous in the treatment or prevention of diseases potentially occurring at the same time and/or in the same organ so that the potential occurrence of one or more distinct targets is predictable or likely. Thus, the basis for designing a polycomposition according to the present invention is good knowledge about relevant targets and their potential occurrence in certain situations or under certain conditions. Relevant disease-related targets are widespread and may be causing diseases directly (primary diseases), or may be the cause of diseases which are secondary to primary diseases. Relevant targets also include exogenous pathogenic agents to which an individual may have been or is likely to become exposed to.

The present invention provides a polycomposition for use in prophylaxis or therapy of an individual in risk of suffering of one or more primary diseases or secondary infections commonly associated with primary diseases; and for which a prophylaxis or therapy may be desirable. A suitable polycomposition may be directed against the most common distinct targets associated with the primary disease or disease complex. Targets associated with primary diseases are described below.

Furthermore, targets associated with infections that are secondary to primary diseases such as other infections, cancer, transplantation, splenectomy, surgery, multiple trauma and burns will be described below. The available information about distinct targets relating to a particular disease or disease complex may be used to design a polycomposition according to the invention for use in the prophylaxis and therapy of said secondary diseases or disease complexes.

A polyclonal antibody typically has at least 2, 3, 4, 5, 6, 8, 10, 20, 30, 40, 50, 100, 1000 or 10⁴ distinct members. In a preferred embodiment of the invention each set of target-specific recombinant polyclonal antibodies included in the polycomposition has at least 2 distinct members, preferably at least 3 distinct members, for example at least 4 distinct member, more preferably at least 5 distinct members. The number of distinct members in each set of target specific recombinant polyclonal antibodies is determined by the number and the complexity of the antigen(s) associated with the target and the variability of the antigen(s). It is to be understood that different sets of target-specific recombinant polyclonal antibodies may comprise different numbers of distinct members.

### Primary Diseases

The polycomposition according to the present invention is particularly advantageous in the treatment or prevention of primary diseases potentially occurring at the same time and/or in the same organ so that the potential occurrence of one or more distinct targets is predictable or likely. Relevant disease-related targets associated with primary diseases are plenty and include infectious pathogens, allergens, biowarfare agents, toxins, venoms, etc. as described in the following:

### Primary Infectious Diseases

The present invention provides methods for broad spectrum prevention and treatment of infections caused by infectious microorganisms e.g. affecting the same organ, such as respiratory infections caused by Respiratory Syncytial virus, Parainfluenza virus and Human Metapneumovirus. In the context of the present invention these three infectious agents are considered as causing different diseases although the clinical symptoms of an infection by either are similar. These viruses are examples of respiratory infections that may not be easily discriminated, while typically occurring during the same time of the year e.g. during the winter season, and thus a broad-spectrum recombinant polyclonal antibody polycomposition may be desirable for treatment or prevention of premature infants, the elderly, immunocompromised patients, recipients of transplants etc. The present invention provides basis for an improvement in the prevention and treatment of severe respiratory infections where there is a range of possible pathogenic agents as the underlying cause of the infection.

In a similar way, infections of the eye, e.g. keratitis and conjunctivitis, can be caused by a number of agents, which are known but not easily discriminated. Therefore patients suffering from an infection of the eye may be treated by topical administration of a polycomposition of the invention targeting a broad selection of the typical infectious agents of the eye.

### Allergy

Another field in which polycompositions according to the present invention may play an important role is in the prevention and treatment of multiallergic individuals especially individuals staying in an allergen-loaded environment. Some of the allergens might be known to the individual but it is also likely that there will be allergens unknown to the individual or where the presence of the allergens can be predicted with a high degree of certainty from information about the environment, e.g. on a farm. Thus, from knowledge about the general occurrence of allergens in a farm environment, a person skilled in the art will be able to design a suitable polycomposition according to the present invention. This will apply to any allergen-loaded environment where it may be desirable to protect a multiallergic individual staying in this environment against a multiallergic reaction.

In this connection allergy to a particular allergen is considered as one disease and allergy to a different distinct allergen is considered as another disease.

### Asthma and Other Respiratory Diseases

Asthma is a syndrome characterized by variable airway obstruction and inflammation. Asthma is often related to infections, especially viral infections and the aeroallergen sensitization often occurs early in life.

A polycomposition according to the invention for use in prophylaxis or therapy of allergy may comprise antibody activities against virus, bacteria, and possibly other relevant pathogenic microorganisms or any combination thereof.

### Exogenous Pathogenic Agents and Targets in an Environmental Niche

Many diseases are related to exposure to exogenous pathogenic agents, where the composition of the agent (the pathogenic agent is here synonymous with “target”), is not known in detail and where a diagnosis is not yet available. From the knowledge about exogenous disease-causing agents or targets in a particular environment it is often possible to predict the presence of targets for antibodies that might be relevant in the actual situation. From this information it is possible to design a polycomposition according to the present invention comprising two or more sets of recombinant polyclonal antibodies where antibodies of each set are capable of binding to a distinct exogenous disease-causing target or agent. This design concept can be used in relation to a number of environmental niches, such as a the hospital environment, a food-related environment, a water-related environment, sources of allergens, a potential or actual site of biological warfare or bioterrorism, or a site with potential or actual exposure to pathogenic, toxic, potentially pathogenic, or potentially toxic agent.

### Targets Related to a Hospital Environment or Nursery Home

Hospitalization is one of the major risk factors for exposure to harmful agents such as pathogenic microorganisms that may be harmful not only to a patient but also to the hospital staff.

Hospital infections (nosocomial infections) are the result of three factors occurring in tandem: High prevalence of pathogens, efficient mechanisms of transmission and a high prevalence of people at risk of acquiring complicated infections. These factors lead not only to higher transmission of pathogens within the hospitals, but also fast evolution of pathogenicity (enhanced disease-causing potential) among microorganisms in hospitals. Isolation of the patient to control transmission may not be sufficient to protect against spreading of the infection and measures against such infections are very expensive to society.

The hospital environment includes all aspects of the hospital, including the air, all surfaces of equipment, intravenous devices, broken tissue or mucosal barriers, as well as burns and all individuals present in the hospital.

### Targets Related to Foodborne Infections

Food-related infections play an important role even with today's focus on hygiene and control of the food in itself. It is also important to survey the places where food is produced for decreasing the risk of food borne infections.

A polycomposition according to the invention may be designed for use in the treatment or prophylaxis of food-related infections including for use in individuals exposed at places where food is produced, tested or analyzed, or where food is served. The polycomposition may also be used for diagnosis.

### Targets Related to Waterborne Infections

Waterborne nosocomial infections play an important role especially among immunosuppressed patients (e.g. transplant patients, cancer patients), immunocompromised patients (e.g., surgical patients, patients with underlying chronic lung diseases, dialysis patients), and elderly persons). Thus, a polycomposition according to the invention may be designed for use in treatment or prevention of waterborne nosocomial infections.

Water contaminated with waterborne infectious agents is a threat to individuals consuming or coming in contact with such water. This will also apply to water used for food preparation; water used for other purposes where contact with an individual may cause health problems as well as devices having been or which will come in contact with said water. Waterborne infections are also a threat to drinking water supplies, recreational waters, source waters for agriculture and aquaculture. Waterborne pathogens can pose significant human health threats as several outbreaks of infectious diseases associated with drinking water have shown. Thus, a polycomposition according to the invention may be designed from available data on waterborne infections for use in treatment or prevention of waterborne infections commonly seen in a specific geographically area or generally associated with waterborne infections associated with drinking water, water used for food preparation, water used for other purposes where contact with an individual may cause health problems as well as any devices having been or which will come in contact with said water, as well as natural water reservoirs and streams.

Targets Associated with a Site of Potential Biological Warfare or Bioterrorism

The increased risk of bioterrorism all over the world using harmful biological material is requiring new ways of thinking in dealing with all the problems associated with biological warfare. After an actual incident involving maybe hundreds of people, any delay of treatment may be fatal. The relevant sites of biological warfare or bioterrorism include geographical regions where there is an increased risk of biological warfare or bioterrorism or an actual incident has just occurred. A polycomposition according to the present invention comprising recombinant polyclonal antibodies to a number of the most commonly used biological weapons such as anthrax toxin, botulinum neurotoxins, ricin toxin and staphylococcal enterotoxins will be extremely useful for treating or preventing disease in individuals that are expected to have been exposed to the biological weapon. The present polycomposition can be applied without waiting for a diagnosis or the polycomposition may be used in a diagnostic test before the therapy is initiated to ensure that the polycomposition in fact does contain recombinant polyclonal antibodies binding to targets in the biological weapon. Thus, the present invention provides means for immediate action just after an actual incident has occurred.

Polycompositions according to the present invention will be very useful biodefense agents since the use of passive immunotherapy against anthrax, hemorrhagic viruses, botulinum neurotoxins, plaque, tularemia, smallpox or vaccinia virus has shown promising results in animal models or in humans (Bregenholt et al. 2004, Vaccines & Antibodies, 4(3):387-396). A pharmaceutical polycomposition according to the invention could also be administered in combination with vaccines to combine immediate protection with long term pathogen immunity. In addition the present polycompositions can be used together with antibiotics to afford broad-spectrum microbial neutralization following exposure to hard-to-treat pathogens. Thus the polycompositions according to the invention are very attractive remedies in both military and civilian defense against biowarfare agents.

The early detection and diagnosis of infection with a biological warfare agent is essential if intervention is to occur at a point in time where the prognosis can still be influenced and also to guide the selection of the optimum therapeutic protocol. In table 4 the requirement for rapid diagnosis is illustrated. Source: Burnett et al. 2005, Nature Reviews, Drug Discovery, 4, 281-297.

TABLE 4

| Biowarfare | Incubation | | | Time for | |
|---|---|---|---|---|---|
| agent | period | | Disease duration | diagnosis | |
| Anthrax | 1-6 | days | Death in 3-5 days | 18-24 | h |
| | | | (untreated) | | |
| Botulism | 1-5 | days | Death in 24-72 h | 3-21 | days |
| | | | (untreated) | | |
| Plague | 2-3 | days | 1-6 days (usually fatal) | 2 | days |
| Smallpox | 7-17 | days | 4 weeks | 24-48 | h |
| Tularemia | 1-21 | days | >2 weeks | 3 | days |
| Ebola | 4-21 | days | 7-16 days (usually fatal) | 1-3 | days |
| Marburg | 9-10 | days | 5-14 days (usually fatal) | 1-3 | days |
| Brucellosis | 5-60 | days | from >8 weeks to >1 year | 14-21 | days |
| Glanders | 10-14 | days | 7-10 days | 1-3 | days |
| Q Fever | 10-40 | days | 2-14 days | 7-14 | days |
| Viral | 2-6 | days | 2-21 days | 1-3 | days |
| encephalitides | | | | | |
| Ricin toxin | 18-24 | h | 1-12 days | 1-5 | days |

One of the factors that make biowarfare difficult to counter medically is the necessity of a very fast diagnosis. This may often imply that a number of separate diagnostic tests will have to be set up immediately. A diagnostic polycomposition according to the present invention may comprise antibodies reacting with several of the biowarfare agents for use as a first screening means. A polycomposition for use in therapy may comprise antibodies reacting with two or more the most fatal biowarfare agents, such as botulism, anthrax, plaque, Ebola or Marburg. As the incubation time is very short for the ricin toxin, it may be preferred also to include therapeutic antibodies against the ricin toxin. It may also be preferred to include only antibodies reacting with some of these agents. It could also be suggested to make a diagnostic test by combining antibodies reacting with other biowarfare agents having a longer incubation time. By using a screening with relatively few diagnostic tests as mentioned above it may be possible to obtain a very fast overview of the threat faced and the urgency of treatment or prophylaxis.

Targets Associated with a Site of Potential or Actual Exposure to Pathogenic, Toxic, Potentially Pathogenic or Potentially Toxic Substances

If an increased risk of pathogenic or toxic material has been identified, it is important to able to deal with this problem immediately without having to rely on a detailed diagnosis. Based on previous experience it is often possible to predict which pathogenic or toxic material to be expected at a particular site. Thus it is quite often possible to predict the presence of pathogenic microorganisms, microbial toxins, animal toxins or poisonous animals, fungal toxins or poisonous fungal, plant toxins or poisonous plants or drug intoxication.

### Poisonous and Venomous Animals

Poisonous and venomous animals are a significant cause of global morbidity and mortality. Poisonous animals comprises poisonous marine animals and poisonous amphibians. Poisonous dart frogs and poisonous salamanders are examples of poisonous amphibians that may cause severe problems for an individual getting in touch with these animals. In a geographic area where the presence of these amphibians can be expected, a polycomposition according to the present principles may be designed for use in treatment or prevention of the affected individuals.

Venomous animals comprise venomous mammals, venomous insects, venomous marine animals, venomous reptiles and venomous arachnids. A polycomposition according to the present principles may be designed for use in treatment or prophylaxis of individuals affected by a sting of an insect not identified in detail but belonging to the group of venomous insects expected in a particular geographic area. A polycomposition according to the present principles may be designed for use in treatment or prophylaxis of individuals affected by a sting of a venomous marine animal not identified in detail but belonging to the group of venomous marine animals expected in a particular geographic area.

Venomous snakes are found throughout the world, including many oceans, and have evolved a variety of highly effective toxins and methods of delivery. Particularly in the rural tropics, snakebite morbidity and mortality has a significant human medical and economic toll.

The actual mix of toxins in the venom of a given species of snakes may vary individually and also by age and season. Anti-venoms are economically marginal and are becoming less available in many regions. Thus, there is a need for a replacement of anti-venoms. A polycomposition according to the inventions will be an effective replacement and might be designed according to local needs, or to take into accounts venomous snakes from a larger area. It is important to gather information about the venomous snakes before a polycomposition according to the invention is designed whether this is going to be a locally applicable polycomposition or the polycomposition is intended for use in a greater area, such as a country or a continent.

A polycomposition according to the invention for treating or preventing disease after bites or stings from spiders or scorpions may be designed according to local needs, or taking into account spiders or scorpions from a larger geographically area.

### Drug Intoxication Targets

Drug intoxication is another field in which the polycomposition according to the invention will be very useful. A polycomposition according to the invention for determining drug intoxication may be designed from available drug information.

### Secondary Infections as a Consequence of Underlying Disease

The present invention also provides a polycomposition for use in prophylaxis or therapy of an individual in risk of suffering of one or more secondary infections commonly associated with primary diseases for which a prophylaxis or therapy may be desirable. A suitable polycomposition may be directed against distinct targets associated with the most common secondary diseases or disease complexes. Targets associated with infections that are secondary to primary diseases or conditions such as other infections, cancer, transplantation, splenectomy, surgery, intravenous devices, assisted ventilation, multiple trauma and burns will be described below. The available information about distinct targets associated with such secondary infections may be used to design a polycomposition according to the invention for use in the prophylaxis and therapy of said secondary infection. Often, the primary disease causes an increased risk of several secondary infections and thus it is especially advantageous to be able to administer prophylaxis towards or treat two or more secondary infections in the patient or individual, and thus the present invention provides polycompositions for use in prophylaxis or therapy of such an individual in risk of suffering from one or more secondary infections commonly associated with a primary disease for which a prophylaxis or therapy may be desirable. Occasionally, it is desirable to treat or administer prophylactically polyclonal antibody medicines which are directed both against one or more secondary diseases, as well as against a target associated with the primary disease agent.

### Infections Secondary to Acquired Immunodeficiency Syndrome (AIDS)

Suppressed immune responses in HIV-infected patients suffering from AIDS create an advantageous milieu for a unique constellation of pathogens, which include reactivation of latent infections as well as a range of additional infections. The majority of these infections are considered opportunistic; however infections that also occur in patients that are not immunodeficient may cause disease at a greater frequency or severity in HIV patients.

With the improved treatment of HIV, secondary infections play an increased role in the treatment of an HIV-infected individual over time. Thus, any improvement in the treatment or prevention of the secondary infections will be of great importance.

### Secondary Infections Frequently Observed in Cancer Patients

Cancer patients may be susceptible to infections for various reasons. A cancer patient can be immune compromised because of the underlying malignancy itself or due the anti-neoplastic therapy. Certain malignancies directly implicate the cells of the immune system and for example patients with Hodgkin's and non-Hodgkin's lymphoma have abnormalities in the cellular immune response which makes them more susceptible to viral and fungal infections. In solid cancers, infections can typically occur at the site of the tumor. The nature of the infectious agents that invade the host after anti-neoplastic therapy is highly dependent on the intensity of the chemotherapeutic regimen and the duration of the neutropenic period.

In general, patients with solid tumors become less immune compromised than patients with leukemias and lymphomas. This primarily relies on the fact that the immune system itself is not malignantly transformed, and secondly, most chemotherapeutic regimens used for the treatment of solid cancers cause a shortened neutropenic period as compared to the neutropenia observed in hematological cancers. Obviously, in solid tumors, the primary site of the cancer may sometimes be infected and can give rise to a systemic infection. As for all cancers, the infectious spectrum encountered is very much dependent on the presence or absence of an intravenous catheter.

### Secondary Infections Related to Transplantation

All bone marrow transplant patients require immunosuppressive therapy which while controlling rejection, can produce serious side effects including infection and malignancy. The chronic risk of such infection secondary to immunosuppression and resulting immunodeficiency with its diagnostic challenges and potentially fatal outcome requires focus on transplant-associated infections.

During the pre-engraftment period infections are predominantly due to conditioning chemotherapy and will be similar to those seen in other regimens with high-dose chemotherapy causing severe neutropenia. The post-engraftment period is the period from neutrophil recovery until approximately day 100 when B- and T-cell recovery is apparent. In this period, T-cell function may well be blunted by graft versus host disease (GVHD) or immunosuppressive therapy (corticosteroids, cyclosporine, anti-T lymphocyte therapy and ganciclovir). Infections in the post-engraftment period are commonly both due to defect mechanical barriers because of GVHD and due to impaired cellular immunity. The late risk period begins from day 100 and ends when the patients have regained normal immunity, but is of course highly dependent on whether or not the patients becomes free of chronic GVHD that will require continuous immunosuppression. Late infections occur more frequently in patients transplanted with unrelated donors.

Secondary Infections Associated with Intravenous Devices

The carriers of intravenous implants are highly susceptible to bacteremia and other infections. This category of patients is growing, and may carry an intravenous device for a long time, including catheters placed to allow long term central venous access such as Hickman catheters, total parenteral nutrition catheters, totally implanted intravascular access devices, balloon-tipped pulmonary artery catheters, and arterial lines.

In large studies, device-associated infections have been shown to account for almost half of all nosocomial infections. Source: Mandell, Douglas and Bennett's “Principles and Practice of infectious Diseases, Vth edition, chapter 292, 3015-3016, references 1-5.

Bacteria get access to the intravascular device not only via the skin, but also via alternative ports in the infusion fluids, among which for example parenteral nutrition creates a superb substrate for microbes.

As described above, neutropenic patients are at a special risk of IV-device infection, and also in this patient category IV device-associated infections are the most common causes of bacteremia. Not only neutropenic patients are at risk of IV-catheter infection. Other risk groups are individuals less than one or more than 60 years of age, patients achieving immunosuppressive therapy, patients with loss of skin integrity, with severe underlying conditions and with other (distant) infections.

### Secondary Infections Related to Splenectomy

Splenectomized patients have long been known to be susceptible to fatal infectious disease, particularly caused by encapsulated bacterial organisms. Furthermore, many conditions where splenectomy has not been performed may render the patient functionally hypo- or asplenic, and thus render these patients prone to serious infections.

Splenectomy may be performed for many reasons—in trauma, during surgery for further reasons, in hemolytic anemia or idiopathic thrombocytopenic purpura (ITP) where medical treatment fails, and in the treatment of some splenic lymphomas (particularly splenic marginal zone lymphomas).

Functional hyposplenism is seen in many conditions including: autoimmune conditions (e.g. rheumatoid arthritis, Hashimotos thyroiditis, lupus erythematosus), hematological diseases (e.g. sickle cell anemia, thalassemia), neoplasia (e.g. lymphoma), infiltrative diseases (e.g. amyloidosis, sarcoidosis), intestinal disorders (e.g. Crohn's disease, celiac disease) and other conditions such as in the elderly >70 years.

### Secondary Infections Related to Surgery

The susceptibility to wound infection during surgery may be related to (I) the surgical procedure itself including the hygienic conditions and the use of foreign implants II) the immune status of the patient, nutritional condition and concomitant diseases such as diabetes, and (III) the use of antimicrobial prophylaxis during and after surgery.

Bacterial contamination during surgery is inevitable although aseptic techniques have improved considerably. The actual load of microbes seems to be crucial for whether or not an infection may settle and give rise to secondary sepsis. Numerous species have been described in wound infection after surgery.

### Secondary Infections Related to Multiple Trauma

Trauma patients are at increased risk of infection for a number of reasons. Broken tissue and mucosal barriers allow access of contaminating pathogens to the blood steam. Ischemic tissue or devitalized tissue may be colonized by microbes, and as mentioned above the use of numerous intravenous devices further allow pathogens to enter the blood stream. Once colonized, wounds drains etc. cannot be cleared of infection by the host immune defense. Many of these patients will encounter the common problems related to long term intensive care which may further increase their susceptibility to infections including tracheotomies, immobilization, bedsores, intravascular monitoring etc.

### Secondary Infections Related to Burns

The risk of infection in burns relates directly to the extent of the injury, and the primary treatment is concentrated on limiting the area of injury by supplying oxygen, nutrition and circulation to the wound. The removal of non-viable tissue is another crucial task. During such wound manipulation early intervention with prophylactic antibiotics may save patients from serious infections. Again cutaneous microbes are among the most commonly seen, however the spectrum of infectious agents may be related both to the patients' normal flora and to the specific flora in the individual burn-ward.

In a similar way, chronic wounds, e.g. those experienced by many diabetics, are also subject to infection by cutaneous microbes and the exact type of infection may be difficult to determine. Therefore chronic wounds may also benefit from treatment with a broad-spectrum polycomposition of the present invention.

### Supplementary Active Ingredients

A polycomposition according to the invention may be provided together with one or more monoclonal antibodies or immunoglobulin. In case it is advantageous to administer the polyclonal antibody product together with a vaccine, the two or more products may be delivered in a kit in separate containers.

Polycompositions used for diagnosis, prophylaxis and treatment may be designed and used in parallel.

Therapeutic or prophylactic polycompositions should comprise medically efficacious amounts of each of the different sets of recombinant polyclonal antibodies for obtaining the desired clinical effect.

### Design Parameters

Based on the knowledge about the characteristics of responses against specific targets, a number of parameters should be evaluated when designing target-specific recombinant polyclonal antibodies. Firstly, the impact of the natural antibody response in vivo should be considered by analyzing the antibody repertoires from individuals recovering from effects related to the relevant target. Secondly, the neutralizing potential of antibodies raised against individual epitopes should be dissected in order to identify potentially neutralizing epitopes and avoid using working capacity by including antibodies against epitopes with little or no impact. Thirdly, the antibody repertoire should be selected to cover as broad a range of neutralizing epitopes as possible, while maintaining an antibody composition resembling the neutralizing host immune response as closely as possible. Lastly, the recombinant polyclonal antibodies should be produced with desirable effector functions reflecting those relevant in the natural antibody-mediated elimination. Use of different recipient vectors containing sequences for different constant regions which could be □1, □2, □3, □4, □, □1, □2, □ or □ provides a very easy way of controlling the effector function of the composition according to the invention. The use of different expression systems, which could be cells of plant, bacterial, eukaryotic or fungal origin (also genetically manipulated) likewise provide means for modulating the glycosylation pattern of the antibodies and hence their effector function.

Isolation and Selection of Variable Heavy Chain and Variable Light Chain Coding Pairs from Each Distinct Target

The process of generating each of the two or more sets of target-specific recombinant polyclonal antibody involves the isolation of sequences coding for variable heavy chains (V_{H}) and variable light chains (V_{L}) from a suitable source, thereby generating a repertoire of V_{H} and V_{L} coding pairs. Generally, a suitable source for obtaining V_{H} and V_{L} coding sequences are lymphocyte containing cell fractions such as blood, spleen or bone marrow samples from one or more individuals having reacted to a relevant target with a suitable immune response. Preferably, lymphocyte containing fractions are collected from humans or transgenic animals with human immunoglobulin genes having reacted to the relevant target. The collected lymphocyte containing cell fraction may be enriched further to obtain a particular lymphocyte population, e.g. cells from the B lymphocyte. Preferably, the enrichment is performed using magnetic bead cell sorting (MACS) and/or fluorescence activated cell sorting (FACS), taking advantage of lineage-specific cell surface marker proteins for example for B cells and/or plasma cells. Preferably, the lymphocyte containing cell fraction is enriched with respect to B cells and/or plasma cells. Even more preferred cells with high CD19 and CD38 expression and intermediate CD45 expression are isolated from blood. These cells are sometimes termed circulating plasma cells, early plasma cells or plasma blasts, for ease, they are just termed plasma cells in the present invention.

The isolation of V_{H} and V_{L} coding sequences can be performed in any way, where the V_{H} and V_{L} coding sequences are combined in a vector to generate a library of V_{H} and V_{L} coding sequence pairs. In the classical way, the V_{H} and V_{L} coding sequences are combined randomly in a vector to generate a combinatorial library of V_{H} and V_{L} coding sequences pairs. The isolation of V_{H} and V_{L} coding sequences can e.g. be performed by phage display and hybridoma technology including use of transgenic animals.

However, in the present invention it is preferred to mirror the diversity, affinity and specificity of the antibodies produced in a humoral immune response upon challenge with a relevant target. This involves the maintenance of the V_{H} and V_{L} pairing originally present in the donor, thereby generating a repertoire of sequence pairs where each pair encodes a variable heavy chain (V_{H}) and a variable light chain (V_{L}) corresponding to a V_{H} and V_{L} pair originally present in an antibody produced by the donor from which the sequences are isolated. This is also termed a cognate pair of V_{H} and V_{L} encoding sequences and the antibody is termed a cognate antibody. Preferably, the V_{H} and V_{L} coding pairs of the present invention, combinatorial or cognate, are obtained from human donors, and therefore the sequences are completely human.

There are several different approaches for the generation of cognate pairs of V_{H} and V_{L} encoding sequences, one approach involves the amplification and isolation of V_{H} and VL encoding sequences from single cells sorted out from a lymphocyte-containing cell fraction. The V_{H} and V_{L} encoding sequences may be amplified separately and paired in a second step or they may be paired during the amplification (Coronella et al. 2000 Nucleic Acids Res. 28: E85; Babcook et al 1996 PNAS 93: 7843-7848 and WO 2005/042774). An alternative approach involves in-cell amplification and pairing of the V_{H} and V_{L} encoding sequences (Embleton et al. 1992. Nucleic Acids Res. 20: 3831-3837; Chapal et al. 1997 BioTechniques 23: 518-524). In order to obtain a repertoire of V_{H} and V_{L} encoding sequence pairs which resemble the diversity of V_{H} and V_{L} sequence pairs in the donor, a high-throughput method with as little scrambling (random combination) of the V_{H} and V_{L} pairs as possible, is preferred, e.g. as described in WO 2005/042774 (hereby incorporated by reference).

Preferably a repertoire of V_{H} and V_{L} coding pairs, where the member pairs mirror the gene pairs responsible for the humoral immune response upon challenge with a target is generated according to a method comprising the steps i) providing a lymphocyte-containing cell fraction from one or more donors having reacted to a relevant target; ii) optionally enriching B cells or plasma cells from said cell fraction; iii) obtaining a population of isolated single cells, comprising distributing cells from said cell fraction individually into a plurality of vessels; iv) amplifying and effecting linkage of the V_{H} and V_{L} coding pairs, in a multiplex overlap extension RT-PCR procedure, using a template derived from said isolated single cells and v) optionally performing a nested PCR of the linked V_{H} and V_{L} coding pairs. Preferably the isolated cognate V_{H} and V_{L} coding pairs are subjected to a screening procedure as described below.

Once the V_{H} and V_{L} sequence pairs have been generated, a screening procedure to identify sequences encoding V_{H} and V_{L} pairs with binding reactivity towards a relevant target is performed. The screening for binders to a target is generally performed with immunodetection assays such as FACS, ELISA, FLISA and/or immunodot assays.

The V_{H} and V_{L} pair encoding sequences selected in the screening are generally subjected to sequencing, and analyzed with respect to diversity of the variable regions. In particular the diversity in the CDR regions is of interest, but also the V_{H} and V_{L} family representation is of interest. Based on these analyses, sequences encoding V_{H} and V_{L} pairs representing the overall diversity of the agent-binding antibodies isolated from one or more donors are selected. Preferably, sequences with differences in all the CDR regions (CDRH1, CDRH2, CDRH3 and CDRL1, CDRL2 and CDRL3) are selected. If there are sequences with one or more identical or very similar CDR regions which belong to different V_{H} or V_{L} families, these are also selected. The selection of a V_{H} and V_{L} sequence pairs can also be performed based on the diversity of the CDR3 region of the variable heavy chain. During the priming and amplification of the sequences, mutations may occur in the framework regions of the variable region. Preferably, such errors are corrected in order to ensure that the sequences correspond completely to those of the donor, e.g. such that the sequences are completely human in all conserved regions such as the framework regions of the variable region.

When it is ensured that the overall diversity of the collection of selected sequences encoding V_{H} and V_{L} pairs is highly representative of the diversity seen at the genetic level in a humoral response to a challenge with a distinct target, it is expected that the overall specificity of antibodies expressed from a collection of selected V_{H} and V_{L} coding pairs, also are representative with respect to the specificity of the antibodies produced in the challenged donors. The degree of specificity correlates with the number of different epitopes towards which binding reactivity can be detected. The specificity of the individual antibodies expressed from a collection of selected V_{H} and V_{L} coding pairs can be analyzed by Western blot. Briefly, the antigens from the relevant target are resolved on polyacrylamide gel, under reducing conditions. The antibodies are analyzed individually in a Western blot procedure, identifying the protein antigens to which they bind. The binding pattern of the individual antibodies is analyzed and compared to the other antibodies expressed from a collection of selected V_{H} and V_{L} coding pairs. Preferably, individual members to be included in a set of recombinant polyclonal antibody are selected such that the specificity of the antibody collectively covers all the antigens of the target which have produced significant antibody titers in a serum sample from the donor(s).

This procedure is repeated for each distinct target generating the nucleic acid sequences encoding all of the recombinant polyclonal antibodies to be included in a given polycomposition according to the invention.

Production of a Polycomposition from Selected V_{H} and V_{L} Coding Pairs

A polycomposition according to the present invention is preferably produced by culturing a mixture of antibody-expressing polyclonal cell lines in one or a few bioreactors. The antibodies of the polycomposition can be purified from the reactor(s) as a single preparation without having to separate the individual antibodies constituting the polycomposition during the process.

A polycomposition according to the invention may be produced by a method which is a modification of the method described in WO 2004/061104 (incorporated herein by reference). The method for the production of a set of target-specific recombinant polyclonal antibody described in WO 2004/061104 is based on site-specific integration of the antibody coding sequence into the genome of the individual host cells, ensuring that the V_{H} and V_{L} antibody chains are maintained in their original pairing during production. Further, the site-specific integration minimizes position effects and therefore the growth and expression properties of the individual cells in the polyclonal cell line are expected to be very similar. Generally, the method involves the following: i) a host cell with one or more recombinase recognition sites; ii) an expression vector with at least one recombinase recognition site compatible with that of the host cell; iii) generation of a collection of expression vectors by transferring the V_{H} and V_{L} coding pairs derived from a donor having reacted to a distinct target related to a particular disease from the screening vector to an expression vector such that a full-length antibody or antibody fragment can be expressed from the vector; iv) transfection of the host cell with the collection of expression vectors and a vector coding for a recombinase capable of combining the recombinase recognition sites in the genome of the host cell with that in the vector; v) obtaining/generating a polyclonal cell line from the transfected host cell and vi) expressing and collecting the polyclonal antibody from the polyclonal cell line.

The present invention provides a method for producing a polycomposition comprising two or more sets of target-specific recombinant polyclonal antibodies in a single batch or a few batches using a modification of the Sympress™ technology described in WO 2004/061104. Other variations of the production method may be used. Preferably, the nucleic acid sequences encoding the target-specific antibodies are isolated as described above using the Symplex™ technology described in WO 2005/042774 (incorporated herein by reference).

A further development of the method described above allows the simultaneous production of two or more sets of target-specific recombinant polyclonal antibodies, where each set of recombinant polyclonal antibody binds to a specific target which is associated with a particular disease which may be treated or prevented by the use of antibodies

Preferably mammalian cells such as CHO cells, COS cells, BHK cells, myeloma cells (e.g., Sp2/0 or NS0 cells), fibroblasts such as NIH 3T3, and immortalized human cells, such as HeLa cells, HEK 293 cells, or PER.C6, are used. However, non-mammalian eukaryotic or prokaryotic cells, such as plant cells, insect cells, yeast cells, fungi, E. coli etc., can also be employed. A suitable host cell comprises one or more suitable recombinase recognition sites in its genome. The host cell should also contain a mode of selection which is operably linked to the integration site, in order to be able to select for integrants, (i.e., cells having an integrated copy of an expression vector or expression vector fragment encoding the antibody in the integration site). The preparation of cells having an FRT site at a pre-determined location in the genome was described in e.g. U.S. Pat. No. 5,677,177. Preferably, a host cell only has a single integration site, which is located at a site allowing for high expression of the integrant (a hot-spot).

A suitable expression vector comprises a recombination recognition site matching the recombinase recognition site(s) of the host cell. Preferably the recombinase recognition site is linked to a suitable selection gene different from the selection gene used for construction of the host cell. Selection genes are well known in the art, and include glutamine synthetase gene (GS) and neomycin. The vector may also contain two different recombinase recognition sites to allow for recombinase-mediated cassette exchange (RMCE) of the antibody coding sequence instead of complete integration of the vector. RMCE is described in (Langer et al 2002; Schlake and Bode 1994). Suitable recombinase recognition sites are well known in the art, and include FRT, 10× and attP/attB sites. The constant region encoding sequences may be provided by the integrating vector and may include introns. Preferably, the integrating vector is an isotype-encoding vector. Preferably the constant region encoding sequences is present in the vector prior to transfer of the V_{H} and V_{L} coding pair from the screening vector.

The effector function being controlled by the constant region of the antibodies may be designed to be identical or different. The sequence encoding the constant region of an antibody is in the present invention preferably present in the vector to which the sequences encoding the variable sequences of the antibody are transferred (the recipient vector) thereby creating the expression vector. Use of different recipient vectors provides a very easy way of controlling the effector function of the composition according to the invention.

The constant regions present in the vector can either be the entire heavy chain constant region (CH₁ to CH₃ or to CH₄) or the constant region encoding the Fc part of the antibody (CH₂ to CH₃ or to CH₄). The light chain Kappa or Lambda constant region may also be present prior to transfer. The choice of the number of constant regions present, if any, depends on the screening and transfer system used. The heavy chain constant regions can be selected from the isotypes IgG, IgA, IGE, IGM and IgD. One or more of the subtypes IgG1, IgG2, IgG3, IgG4, IgA1, and IgA2 are preferred. More preferred are the isotypes IgG1 and/or IgG3. Further, the expression vector for site-specific integration of the nucleic acid encoding the target-specific antibody contains suitable promoters or equivalent sequences directing high levels of expression of each of the V_{H} and V_{L} chains.

The transfer of the selected V_{H} and V_{L} coding pairs from the screening vector can be performed by conventional restriction enzyme cleavage and ligation, such that each expression vector molecule contain one V_{H} and V_{L} coding pair. Preferably, the V_{H} and V_{L} coding pairs are transferred individually, they may however also be transferred in-mass if desired. When all the selected V_{H} and V_{L} coding pairs are transferred to the expression vector a collection or a library of expression vectors is obtained. Alternative ways of transfer may also be used if desired.

Methods for transfecting a nucleic acid sequence into a host cell are known in the art. To ensure site-specific integration a suitable recombinase must be provided to the host cell as well. This is preferably assured by co-transfection of a plasmid encoding the recombinase. Suitable recombinases are for example FLP, Cre or phage Φ31 integrase, when used together with a host cell/vector system with the corresponding recombinase recognition sites. The host cell can either be transfected in bulk, meaning that the library of expression vectors is transfected into the cell line in one single reaction thereby obtaining a polyclonal cell line. Alternatively, the collection of expression vectors can be transfected individually into the host cell, thereby generating a collection of individual cell lines (producing monoclonal antibodies). The cell lines generated upon transfection (monoclonal or polyclonal) are then selected for site specific integrants, and adapted to grow in suspension and serum free media, if they did not already do this prior to transfection. If the transfection was performed individually, the individual cell lines are analyzed further with respect to their growth properties and antibody production. Preferably cell lines with proliferation rates and antibody expression levels are selected for the generation of the polyclonal cell line. The polyclonal cell line capable of producing a polycomposition according to the invention is then generated by mixing the individual cell lines in a predefined ratio.

As shown in the appended examples, the ratio between the individual polyclonal working cell banks is maintained during culturing. The example shows this for a 50/50 ratio between two polyclonal working cell banks. It is of course conceivable that the polyclonal working cell banks can be mixed in different ratios if it is desired to obtain a polycomposition wherein a polyclonal antibody against one target represents more or less than 50% of the antibodies in the composition. For example two pWCBs may be mixed in any ratio ranging from 1/99 to 99/1, for example 5/95, 10/90, 20/80, 25/75, 30/70, 40/60 or any ratio thereinbetween. Similarly, when three or more pWCBs are mixed to produce a polycomposition comprising three or more sets of target-specific recombinant polyclonal antibodies, then this ratio may be any conceivable ratio among the three constituents. The rationale behind having a ratio different from a 1:1 or 1:1:1 ect ratio may be that one of the sets of antibodies is less potent than the others and therefore needs to be present in higher amounts, or it could be that one of the sets is more important for achieving a clinical benefit, e.g. if this specific set targets a more severe condition.

Generally, a mixed polyclonal master cell bank (mixed pMCB) and/or a mixed polyclonal working cell bank (mixed pWCB) are laid down from a polyclonal cell line expressing two or more sets of target-specific recombinant polyclonal antibodies each set capable of binding to a distinct target wherein said distinct target are related to diseases, which may be treated or prevented by the use of antibodies. It may be advantageous to generate one or more of the pWCB and/or pWCB separately. Thus, the mixed pMCB and/or mixed pWCB are generated from a polyclonal cell line expressing all the antibodies to be included in the polycomposition or the mixed pMCB and/or mixed pWCB may be generated from a polyclonal cell line expressing some of the antibodies to be included in the polycomposition wherein the remaining antibodies are produced from one or more pMCB and/or pWCB added separately before the cells are cultured for expression of all of the antibodies. The polyclonal cell lines may be stored as pMCB for producing a set recombinant polyclonal antibody binding to a distinct target or the polyclonal cell lines may be stored as a mixed pMCB comprising cell lines expressing a mixture of antibodies directed to two or more distinct targets, wherein said distinct targets are related to diseases, which may be treated or prevented by the use of antibodies. The manufacturing of the polycomposition according to the present invention may be starting using a vial containing the relevant mixed pMCB or pWCB or using a mixture of vials each containing a pWCB or pMCB expressing a set of the recombinant polyclonal antibodies to be included in the polycomposition according to the invention.

One embodiment of the present invention is a polyclonal cell line wherein each individual cell is capable of expressing a single V_{H} and V_{L} coding pair, and the polyclonal cell line as a whole is capable of expressing a collection Of V_{H} and V_{L} coding pairs, where each V_{H} and V_{L} coding pair encodes an antibody molecule binding to a distinct target associated with a particular disease, which may be treated or prevented by the use of antibodies. Preferably the collection of V_{H} and V_{L} coding pairs are cognate pairs generated as described above.

The polycomposition is then expressed by culturing one ampoule of a mixed pWCB or by combining and culturing one ampoule from as many pWCB's as it is decided to grow together.

In one embodiment of the method according to the present invention for the manufacture of a composition comprising two or more sets of target-specific recombinant polyclonal antibodies, each set of target-specific recombinant polyclonal antibody capable of binding to a distinct target, wherein said distinct targets are related to diseases, which may be treated or prevented by the use of antibodies, and wherein said composition is essentially free from immunoglobulin molecules that do not bind to one of said distinct targets, said method comprises
providing two or more cell lines wherein each cell line contains a number of different antibody clones capable of expressing unique antibodies,
combining said cell lines in a single container and culturing the cells under conditions facilitating expression of said two or more sets of target-specific recombinant polyclonal antibodies; and
recovering said two or more sets of target-specific recombinant polyclonal antibodies from the cell culture cells or cell culture supernatant.

The polyclonal cell line(s) are cultured in an appropriate medium for a period of time allowing for sufficient expression of antibody and where the polyclonal cell lines remains stable (The window is approximately between 15 days and 50 days). Culturing methods such as fed batch or perfusion may be used. The polycomposition is obtained from the culture medium and purified by conventional purification techniques. Affinity chromatography combined with subsequent purification steps such as ion-exchange chromatography, hydrophobic interactions and gel filtration have frequently been used for the purification of IgG. Following purification, the presence of all the individual antibodies in the polycomposition is assessed, for example by ion-exchange chromatography. The characterization of a polyclonal antibody composition is described in detail in WO 2006/007850 (hereby incorporated by reference).

An alternatively method of expressing a mixture of antibodies in a recombinant host is described in WO 2004/009618, this method produces antibodies with different heavy chains associated with the same light chain from a single cell line. This approach may be applicable if the polycomposition according to the invention is produced from a combinatorial library.

### Pharmaceutical Compositions

An aspect of the present invention is a pharmaceutical composition for therapy or prophylaxis of an individual suffering from one or more diseases the simultaneous treatment or prevention of which is expected to be relevant in a particular situation, e.g. when different cancer types are known to occur in the same organ and/or at the same time in other organs. An example is a pharmaceutical polycomposition comprising recombinant polyclonal antibodies targeting the EGF receptor as well as the vascular endothelial growth factor receptor.

The Pharmaceutical Composition Further Comprises One or more pharmaceutically acceptable excipient.

The present pharmaceutical composition comprising relevant recombinant polyclonal antibodies or polyclonal fragments thereof may be administered within a pharmaceutically-acceptable diluent, carrier, or excipient, in unit dose form. A unit dose form comprises two or more recombinant polyclonal antibodies binding to relevant distinct targets in a therapeutically and/or prophylactically effective amount. The unit dose form according to the present invention may comprise the antibody members of the target-specific recombinant polyclonal antibodies in an amount in the range of 1 μg to 1 g, preferably 1 μg to 1000 μg, more preferably 2-500 μg, even more preferably 5-50 μg, most preferably 10-20 μg.

Conventional pharmaceutical practice may be employed to provide suitable formulations or compositions to administer to individuals to be treated or subjected to prophylaxis. Any appropriate route of administration may be employed, for example, administration may be parenteral, intravenous, intra-arterial, subcutaneous, intramuscular, intraperitoneal, intranasal, aerosol, suppository, topical, or oral administration. For example, therapeutic formulations may be in the form of, liquid solutions or suspensions; for oral administration, formulations may be in the form of tablets or capsules chewing gum or pasta; for topical administration in the form of drops, powders or aerosols; and for intranasal formulations, in the form of powders, nasal drops, or aerosols.

Regardless of the manner of administration, the specific dose may be calculated according to body weight, body surface or organ size. Further refinement of the calculations necessary to determine the appropriate dosage for treatment or prophylaxis involving each of the above mentioned formulations is routinely made by those of ordinary skill in the art. Appropriate dosages may be ascertained through use of appropriate dose-response data.

The pharmaceutical compositions of the present invention are prepared in a manner known per se, for example, by means of conventional dissolving, lyophilizing, mixing, granulating or confectioning processes. The pharmaceutical compositions may be formulated according to conventional pharmaceutical practice (see for example, in Remington: The Science and Practice of Pharmacy (20th ed.), ed. A. R. Gennaro, 2000, Lippincott Williams & Wilkins, Philadelphia, Pa. and Encyclopedia of Pharmaceutical Technology, eds. J. Swarbrick and J. C. Boylan, 1988-1999, Marcel Dekker, New York, N.Y.).

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, pills, or capsules, which may be coated with shellac, sugar or both. It is also possible for them to be incorporated into plastics carriers that allow the active ingredients to diffuse or be released in measured amounts.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, tablets, pills, or capsules. The formulations can be administered to human individuals in therapeutically or prophylactic effective amounts (e.g., amounts which prevent, eliminate, or reduce a pathological condition) to provide therapy for a disease or condition. The preferred dosage of therapeutic agent to be administered is likely to depend on such variables as the type and extent of the disorder, the overall health status of the particular patient, the formulation of the compound excipients, and its route of administration.

The pharmaceutical composition according to the invention may be provided together with one or more monoclonal antibodies, immunoglobulins or other polyclonal antibodies capable of binding to a relevant distinct target in one container or separate containers. Furthermore the pharmaceutical composition according to the invention may be provided together with one or more agents selected from the group consisting of antibiotics, chemotherapeutic agents, activity potentiators, hormones, cytokines, sedatives, analgesics and anti-inflammatory compounds.

A kit for treatment or prevention comprising a pharmaceutical composition according to the invention and optionally one or more monoclonal antibodies, immunoglobulins or other polyclonal antibodies capable of binding to a relevant distinct target may also be provided in separate containers together with two, three or more agents that advantageously could be administered to an individual during the same administration, i.e. shortly before, almost simultaneously or short after administration of the present pharmaceutical composition. This will depend on the condition of the patient and the desired level of treatment or prophylaxis.

### Therapeutic Uses of the Polycompositions According to the Invention

A pharmaceutical composition according to the present invention may be used for prophylaxis or treatment of an individual suffering from one or more diseases which are likely to occur at the same time and/or in the same organ so that a administration of the polycomposition improves the treatment or prophylaxis.

One embodiment of the present invention is a method for treatment or prophylaxis of an individual suffering from different types of cancer, infectious diseases, allergy as well as asthma and other respiratory diseases.

A further embodiment of the present invention is a method for treatment or prophylaxis of an individual who has been or is in risk of being exposed to harmful or potentially harmful targets in a particular environmental niche wherein the targets have not been identified in every detail, but for which sufficient information is available to conclude that the targets potentially are harmful and against which an antibody treatment or prophylaxis may be effective. The particular environment niche may be a hospital environment, a food-related environment, a water-related environment, sources of allergens, a potential or actual site of biological warfare or bioterrorism, or a site with potential or actual exposure to pathogenic, toxic, potentially pathogenic, or potentially toxic agent(s).

A further embodiment of the present invention is the use of a polycomposition according to the invention for the preparation of a pharmaceutically composition for the prophylaxis or therapy of distinct diseases, in particular diseases associated with states of immunodeficiency or immunosuppression.

The pharmaceutically polycomposition according to the present invention may be provided together with further therapeutic agents, optionally is separate containers in a treatment kit.

### Diagnostic Use and Environmental Detection Use

In another aspect of the present invention, the polycomposition is directed to diagnostic use. The polycomposition according to the invention may be use for detecting target agents in the human body as well as in the environment around the human body both being of great importance to human beings as there is a continuous relationship between the human body and the environment. A constant molecular stream connects inside and outside. The antibodies of the polycomposition may be used to capture targets suspected to be present in a given sample. A second detectable antibody may be used to detect the complexes formed between the target and the specific antibodies in a sandwich immunoassay. Although monoclonal antibodies are available for many immunoassays detecting target agents these analyses have been hampered due to inherent difficulties in detecting variations of a target agent. The polycomposition according to the invention will present a great advantage in being able to detect not only different distinct targets in the same assay but also variations of the distinct target agents such as strain variations of a pathogenic microorganism. Thus the present invention is extremely useful for analyzing bulk samples which are not known in details but for which sufficient information is available to conclude that the sample potentially comprises one or more targets from a group of related targets that could be identified with a polycomposition designed to detect such related targets.

In a further aspect of the present invention, the polycomposition is directed to environmental detection use, such as in a food-related or water-related environment or for detecting airborne targets. A food-related environment includes the food as well as places where food is produced, tested or analyzed or where food is served. A water-related environment includes drinking water, water used for food preparation, water used for other purposes where contact with an individual may cause health problems as well as any devices having been or which will come in contact with said water, as well as natural water reservoirs and streams.

The present polycomposition may also be used in forecasting of airborne allergens such as pollen. Such forecasting can determine the optimal timing of prophylaxis or treatment for seasonal allergic reactions. Such compositions may be designed for use in a local area allowing more precise forecasting and hence better possibilities for optimal prophylaxis or treatment.

The polycomposition for diagnostic use may be provided in a diagnostic kit for use in a particular geographically area or a particular environmental niche. Thus, in biological warfare a kit according to the invention may play an important role for early diagnosis and in the guidance for selection of the optimal therapeutic protocol.

### EXAMPLES

An important aspect of the present invention is the co-production of the two or more sets of recombinant polyclonal antibodies to be included in the polycompositions according to the invention in a single or a few batches. This advantaged technology is herein after illustrated by the co-production of two sets of recombinant polyclonal antibodies, one set being capable of binding to Rhesus D antigen, and the other set being capable of binding to the orthopox virus, vaccinia. The example is illustrative for demonstrating the feasibility of co-production of two or more sets of recombinant polyclonal antibodies, wherein each set comprises a number of distinct antibody members binding to epitopes on either the Rhesus D antigen or the vaccinia virus without changing the biological characteristics of the two sets of antibodies in the composition.

### Anti-Rhesus D Recombinant Polyclonal Antibody

Compositions comprising anti-RhD recombinant polyclonal antibody and their use in prophylaxis of hemolytic disease of the newborn (HDN), treatment of idiopathic thrombocytopenic purpura (ITP) and prevention of sensitization to the Rhesus D antigen after mistransfusions of RhD(+) blood to RhD(−) individuals have been described in WO 2006/007850.

The Rhesus blood group antigens are located on transmembrane erythrocyte proteins encompassing the so-called C, c, E, e and D antigens. Approximately 16% of the Caucasian population is Rhesus D negative (RhD(−)) due to an inherited polymorphism. In addition, multiple genetic and serological variants of RhD exist (divided into category II-VII) of which RhDVI is the most clinically relevant.

Polyclonal immunoglobulin preparations against RhD are used worldwide to prevent alloimmunization of pregnant RhD(−) and RhD^{VI}(+) women, thereby preventing HDN. Polyclonal immunoglobulin preparations against RhD (anti-D) are currently obtained by pooling of blood plasma obtained from donors who have become hyperimmune, either through natural RhD alloimmunization or through vaccination of RhD negative volunteer males with RhD positive erythrocytes. The efficacy of anti-RhD immunoglobulin preparations for prophylaxis of HDN is well established and has been in routine use for many years. As a result this severe disease has become a rarity.

Nevertheless the underlying cause of the disease, i.e. alloimmunization of regnant RhD(−) and RhD^{VI}(+) women, still remains and thus requires a continual supply of anti-D immunoglobulin preparations. Further, anti-D immunoglobulin is used after mistransfusions of RhD(+) blood to RhD(−) recipients in order to prevent sensitization to the Rhesus D antigen. The production of an unlimited amount of recombinant anti-RhD polyclonal antibody (anti-RhD rpAb) is described in WO 2006/007850 (incorporated herein by reference).

In short a polyclonal expression cell line producing a rpAb capable of reacting with or binding to Rhesus D antigen was prepared in a multi-step procedure involving the generation of individual expression cell lines (monoclonal cell lines) which each express a unique antibody (WO 2006/007850) and mixing the individual cell lines thereby generating a polyclonal master cell bank (pMCB) from which a polyclonal working cell bank (pWCB) was generated simply by continuing amplification.

### Anti-Vaccinia Recombinant Polyclonal Antibody

The treatment of smallpox outbreaks as a result of bioterrorism and the emergence of related viruses such as monkeypox have revived the need for therapeutics and vaccination against smallpox. Smallpox is caused by airway infection with the orthopox virus, variola. Vaccinia virus is used for vaccination since anti-vaccinia antibodies cross-reacts with variola. Vaccinia vaccination does however mediate moderate to severe adverse reactions in approximately one in every 1000 to one in every 10.000 vaccinated individuals.

Orthopoxviruses produces two types of infectious particles, namely the Intracellular Mature Virions (IMV) and the Extracellular Enveloped Virions (EEV). IMV plays a predominant role in host-to-host transmission and EEV plays a major role in virus propagation within the host. The IMV particle is assembled in the cytoplasm of infected cells and consists of a virally induced membrane surrounding the genome containing a homogenous core particle. EEV particles are generated by wrapping of IMV particles in a host cell-derived membrane followed by egress of the EEV particle. At a later stage the vaccinia virus infection results in cell death and release of the infectious IMV particles. Viral proteins presented at the surface of IMV or EEV particles are potential targets for antibodies, a total of five IMV-specific proteins and two EEV-specific proteins have been reported to elicit virus neutralizing and/or protective effects when used for immunization or vaccination.

Adverse reactions after vaccinia virus vaccination are currently treated with anti-vaccinia virus immunoglobulin (VIG) isolated from donors with a high antibody titer. However, the estimated incidence of adverse effects resulting from a general vaccination program using live attenuated vaccinia virus exceeds the current production capacity of VIG, thereby preventing vaccination as an approach for public protection against smallpox. Production of recombinant anti-vaccinia virus polyclonal antibody (anti-vv rpAb) to be used as an alternative for providing protection against vaccinia virus adverse effects or infections by other orthopoxviruses has been described in PCT/DK2006/000686 published as WO 2007/065433 (incorporated herein by reference).

In short a polyclonal expression cell line producing a rpAb capable of reacting with or binding to vaccinia was prepared in a multi-step procedure involving the generation of individual expression cell lines (monoclonal cell lines) which each express a unique antibody (PCT/DK2006/000686) and mixing the individual cell lines thereby generating a polyclonal master cell bank (pMCB) from which a polyclonal working cell bank (pWCB) was generated simply by continuing amplification.

### Example 1

### Seed Trains for Inoculation of Bioreactors

For the co-production of an anti-RhD rpAb and an anti-vv rpAb, a combination of the two polyclonal working cell banks (pWCB's) described in WO 2006/007850, example 5 (anti-RhD rpAb) and in Example 4 of WO 2007/065433 (anti-vv rpAb), respectively, were used.

To generate the pWCB containing anti-RhD rpAb, one vial of each of 25 banked monoclonal anti-RhD antibody production cell lines had been thawed and expanded. Equal numbers of cells from each culture were then carefully mixed together to generate a pMCB. One vial from this bank was further expanded to generate a pWCB, which was frozen in liquid nitrogen using standard freezing procedures (see details in WO 2006/007850).

To generate the pWCB containing anti-vv rpAb, one vial of each of 28 banked monoclonal anti-vv antibody production cell lines had been thawed and expanded. Equal numbers of cells from each culture were then carefully mixed together to generate a pMCB. One vial from this bank was further expanded to generate a pWCB, which was frozen in liquid nitrogen using standard freezing procedures (see details in PCT/DK2006/000686).

On day 0 (D₀) of the present experiment one frozen ampoule (#7387) of the anti-RhD rpAb pWCB (also termed Sym001 pWCB in the following description) and one ampoule (#12832) of the anti-vv rpAb (also termed Sym002 pWCB in the following description) were thawed in separate T-flasks to recover. Each of the two cell cultures were transferred to separate shaker flask the next day (D₁) for cell propagation and initiation of seed trains for inoculation of bioreactors. Cells in seed train were cultured in ExCell 302 medium with 0.5 mg/mL G418 and with anti-clumping agent (Invitrogen) diluted 1:250 at 37° C., 5% CO₂. Cell number and viability was determined using a Vi-CELL™-cell viability analyzer (Beckman Coulter).

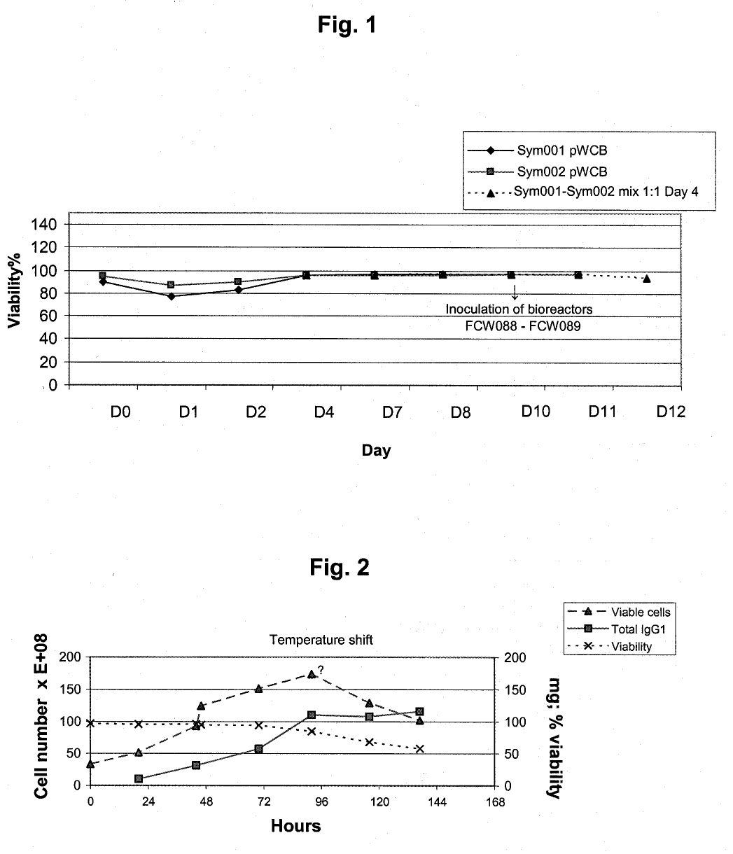

On day 4 (D₄), a mixture of the Sym001 pWCB and Sym002 pWCB (cell number 1:1) was prepared in shaker flasks and cultured together before inoculation on day 10 (D₁₀) in a bioreactor (Experiment # FCW 088). In parallel, the Sym001 pWCB and Sym002 pWCB were kept in separate shaker flasks during the seed train, until day 10 (D₁₀) where a mixture (cell number 1:1) was prepared and inoculated into a bioreactor (Experiment # FCW 089). FIG. 1 shows the viability of the Sym001 pWCB and Sym002 pWCB cultured separately in shaker flasks and Sym001 and Sym002 mixed 1:1 on day 4 and cultured in shaker flasks, respectively. All three cultures showed similar viability prior to inoculation in bioreactors.

On day 10 (D₁₀), one 5-liter bioreactor (B. Braun Biotech International, Melsungen, Germany) was inoculated with 1.34×10⁶ cells/ml from the seed train, which was mixed at day 4 (# FCW 088) and another 5-liter bioreactor (same supplier) was inoculated with a 1:1 mixture of the separately grown Sym001 pWCB and Sym002 pWCB at a concentration of 1.33×10⁶ cells/ml (# FCW 089). The cell cultures were inoculated in 3 liter ExCell 302 medium without G418 and without anti-clumping agent.

The cell numbers and percentage of viable cells in the bioreactors were monitored by Vi-CELL™ analyzer counts during the experiment.

At 46 hours 2 L of ExCell 302 medium was supplemented to the two bioreactors and after 97 hours a temperature downshift from 37° C. to 32° C. was performed. The reactors were cultivated in batch mode and the cell culture supernatant was harvested, when cell viability was ˜55% at 137 hours (D16).

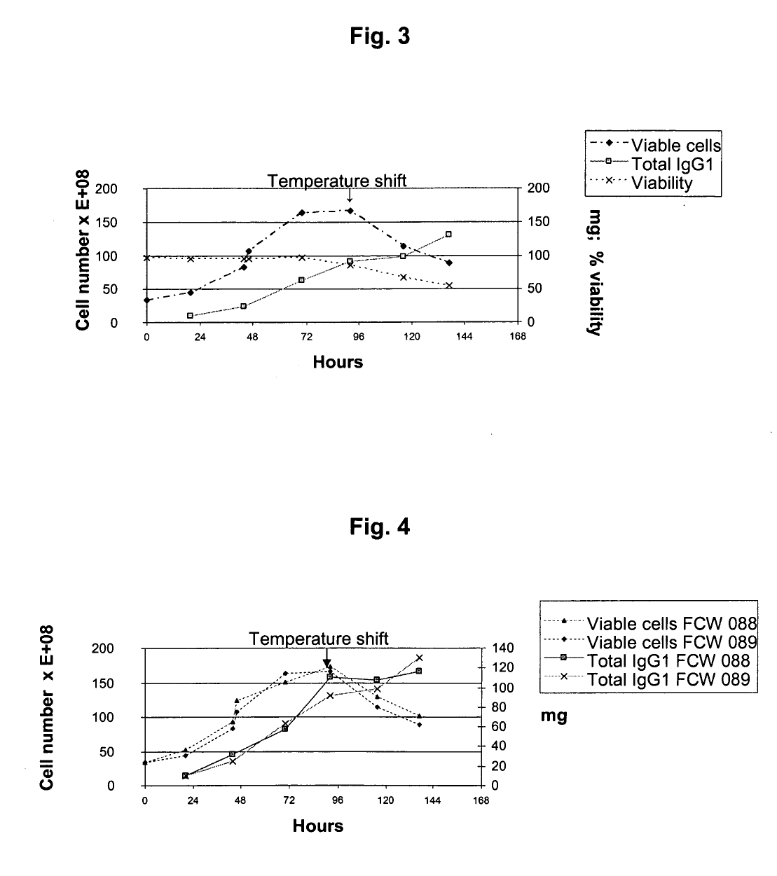

The cell performance in the two bioreactors during the production in batch mode is shown in FIGS. 2-4. FIG. 2 shows the number of viable cells, the viability and the IgG1 titer in the bioreactor in experiment FCW 088, while FIG. 3 shows the same parameters measured in the bioreactor in experiment FCW 089. In FIG. 4, the viable cell number and the IgG1 titer are plotted for both reactors.

The present example demonstrates that a co-production of two recombinant polyclonal antibodies, wherein the two pWCB's were either mixed on day 4 or day 10 of the seed train, were performing alike with respect to cell number, viability and yield when the mixed pWCB's were cultivated as a batch in bioreactors.

The recombinant polyclonal antibodies were obtained from the culture medium in the bioreactors and purified by IgG capture on protein-A columns using conventional purification techniques. Affinity chromatography, either alone or combined with subsequent purification steps such as ion-exchange chromatography, hydrophobic interactions and gel filtration is routinely used for the purification of IgG. The characterization of a polyclonal antibody composition can be performed as described in detail in WO 2006/007853 (hereby incorporated by reference). Following purification, the presence of the individual members in the polyclonal antibody polycomposition was assessed by ion-exchange chromatography, one of the characterization methods described in detail in WO 2006/007853.

Briefly, cation-exchange chromatography separates individual antibody members in a polyclonal antibody composition based on differences in net charge between the individual members and in addition separates forms of individual antibodies that appear charge heterogeneous.

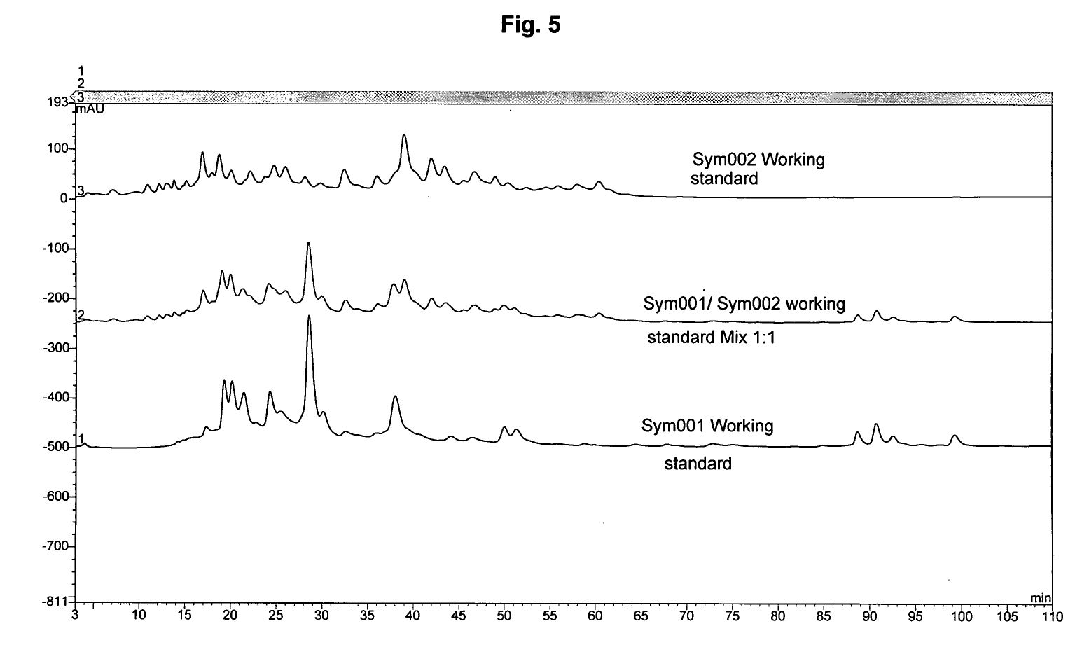

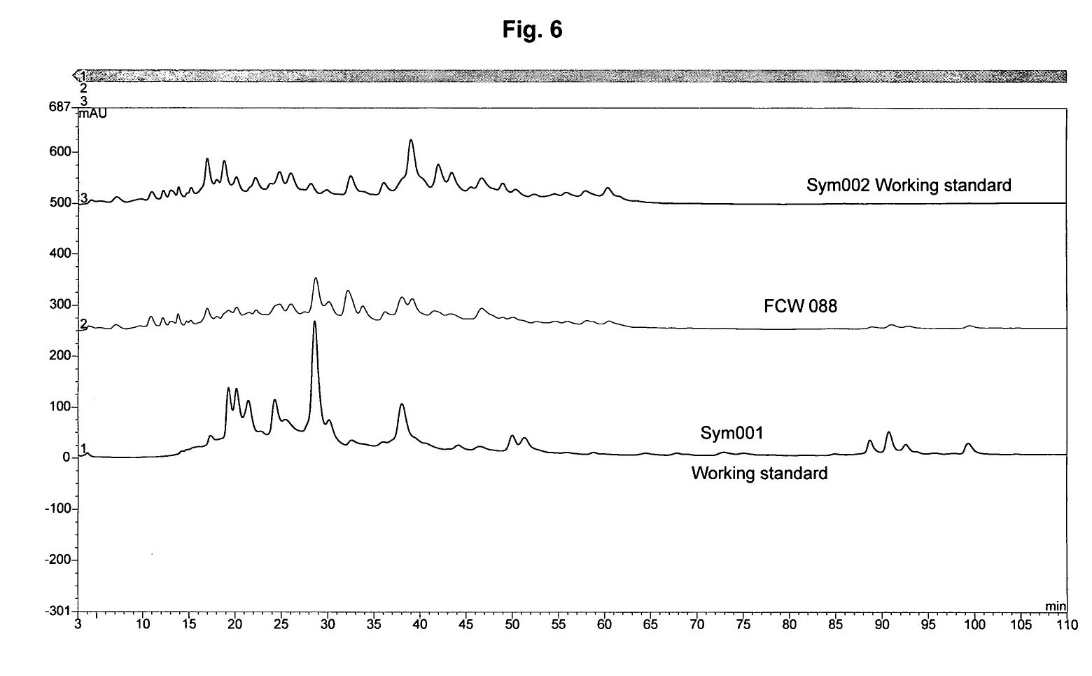

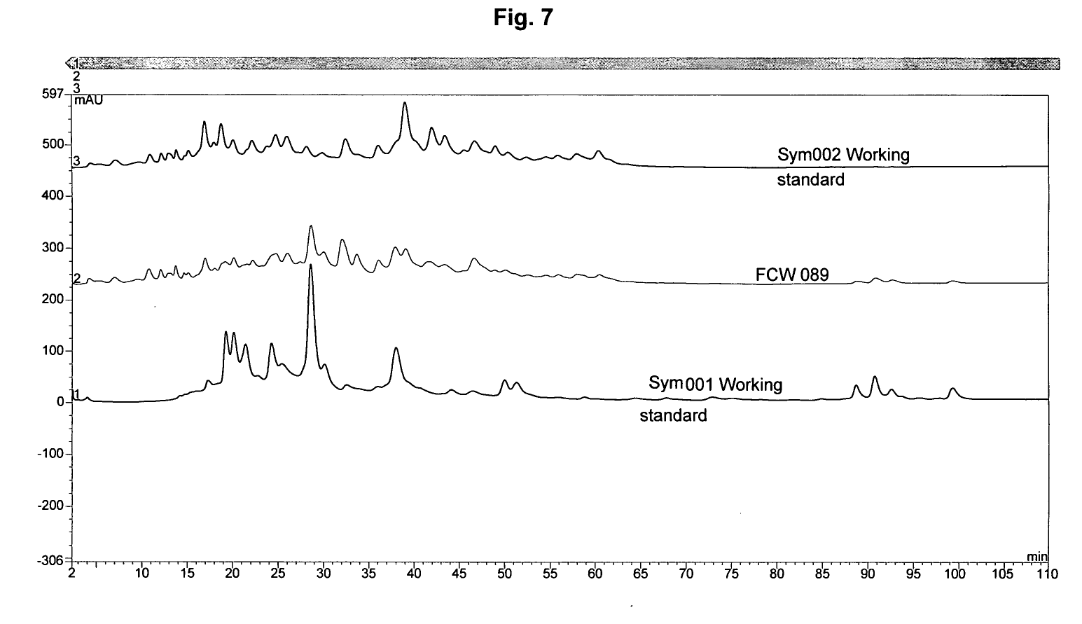

The IEX chromatograms deriving from the co-production of Sym001 and Sym002 in experiments FCW 088 and FCW 089 were compared with IEX profiles from separate Sym001 and Sym002 working standards produced separately, as well as with a 1:1 mix of separately produced Sym001 and Sym002. The resulting data are shown in FIGS. 5-7. It is clearly seen that the FCW088 and FCW 089 IEX profiles contain elements from both Sym001 and Sym002. Each figure is described in detail in the figure legends.

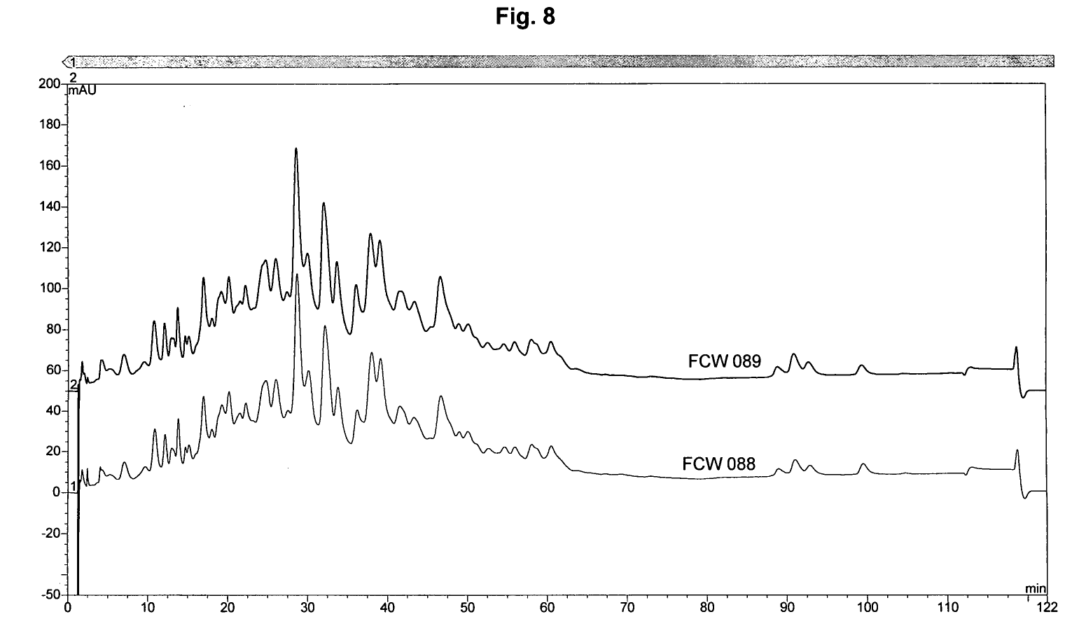

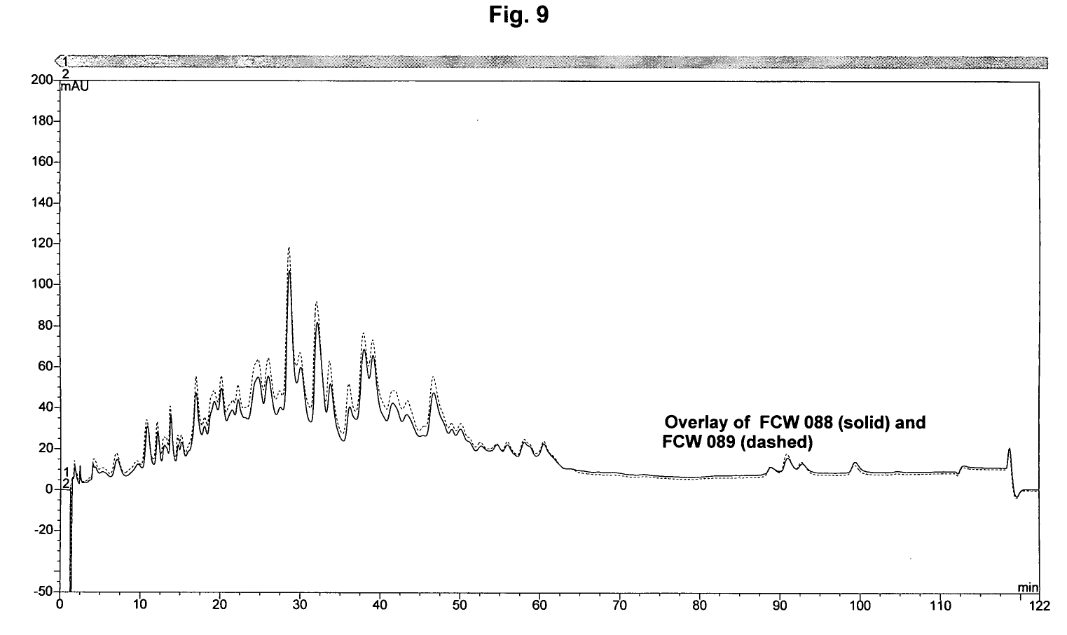

FIGS. 8 and 9 show direct comparisons of FCW 088 and FCW089. Overall, it is clearly shown that the profiles from these two production runs are very similar which indicates that the co-production of Sym001 and Sym002 is reproducible and that the time for mixing cells from the Sym001 and Sym002 pWCB's prior to inoculation of a bioreactor is not critical.

The binding characteristics of the co-produced Sym001 and Sym002 towards RhD-positive erythrocytes and vaccinia virus were examined in order to evaluate any influence from the co-production on the binding characteristics of the two products.

### Potency Assay Using RhD-Positive Erythrocytes

The potency assay used to test for RhD erythrocyte binding activity, which is characteristic for Sym001 was adopted from the European Pharmacopoeia 4 (section 2.7.13 method C) and performed as described in WO 2006/007850, example 6.

The assay results are summarized in Table 18.

Table 18 Potency determination of co-produced polyclonal antibodies from bioreactors FCW 88 and FCW 89 (co-production of Sym001 and Sym002). Units: mfi—mean fluorescent intensity.

TABLE 18
Potency determination of co-produced polyclonal antibodies
from bioreactors FCW 88 and FCW 89 (co-production of
Sym001 and Sym002). Units: mfi-mean fluorescent intensity.

| Sample | Potency | Potency | Potency | Average | Std. Dev. |
|---|---|---|---|---|---|
| 001 WS-100 ng | 147.22 | 228.76 | 199.89 | 191.9567 | 41.34485 |
| 001 WS-200 ng | 365.17 | 453.16 | 410.47 | 409.6 | 44.00145 |
| FCW88-100 ng | 149.89 | 153.99 | 152.61 | 152.1633 | 2.086177 |
| FCW88-200 ng | 338.3 | 324.88 | 330.77 | 331.3167 | 6.726681 |
| FCW89-100 ng | 120.79 | 135.77 | 140.75 | 132.4367 | 10.38912 |
| FCW89-200 ng | 273.84 | 271.39 | 281.33 | 275.52 | 5.178581 |
| 001 + 002-100 ng | 133.35 | 129.8 | 134.56 | 132.57 | 2.474005 |
| 001 + 002-200 ng | 283.87 | 281.33 | 283.87 | 283.0233 | 1.46647 |

“001 WS-100 ng” indicates using a test sample of 100 ng of a Sym001-working standard in the potency assay.
“001 WS-200 ng” indicates using a test sample of 200 ng of a Sym001-working standard.
“FCW88-100 ng” indicates using a test sample of 100 ng of FCW 088.
“FCW88-200 ng” indicates using a test sample of 200 ng of FCW 088.
“FCW89-100 ng” indicates using a test sample of 100 ng of FCW 089.
“FCW89-200 ng” indicates using a test sample of 200 ng of FCW 089.
“001 + 002-100 ng” indicates using a test sample of 100 ng of a 1:1 mix between Sym001 and Sym002 working standards
“001 + 002-200 ng” indicate the result using 200 ng of a 1:1 mix between Sym001 and Sym002 working standards

The activity of Sym001 in FCW 088 and FCW 089 was clearly lower than in the Sym001-Working standards, whereas the two batches of co-produced polyclonal antibodies, FCW 088 and FCW 089 bound to RhD-positive erythrocytes with similar potency.

Further, the activity of these two batches compared well to that of a 1:1 mix of Sym001-Working standard and Sym002-Working standard. This indicates that FCW 088 and FCW 089 contains Sym001 product, and suggests that this component constitutes about 50% of the produced product, rather than 100%.

### ELISA Analysis of FCW 088 and FCW 089 Binding on Vaccinia Virus Antigens

To investigate the two polycompositions, FCW 088 and FCW 089, for vaccinia virus binding activity characteristic for the Sym002 product, each of the polycompositions were analyzed for binding to vaccinia virus antigens by ELISA and compared with an anti-VV rpAb described in PCT/DK2006/000686.

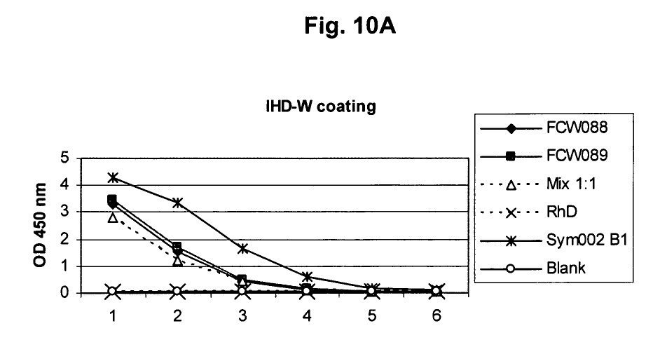

In the ELISA evaluation of FCW 088 and FCW 089, these were compared with Sym002 working standard batch as positive control and Sym001 working standard as negative control, a Sym001/Sym002 working standard mix (1:1) and a blank. The assay was performed using the following vaccinia virus strains: Lister, 1HD-W or vaccinia virus antigens: VCP, B5R A33R and A27L as coatings. The results are shown in FIG. 10.

The present example illustrates that the polycompositions (FCW 088 and FCW 089) performed similar and were comparable with a “Sym001/Sym002 mix (1:1) control” in ELISA using different vaccinia virus antigens as coating.

In summary, this example demonstrates that it is possible to produce a polycomposition consisting of anti-RhD rpAb and anti-vv rpAb in a bioreactor in a reproducible way. Furthermore, it has been demonstrated that the polycomposition showed 50:50 activity against the two distinct targets, RhD antigens and vaccinia virus antigens.

What is claimed is:

1. A composition comprising two or more sets of target-specific recombinant polyclonal antibodies, each set capable of binding to a distinct target, wherein said distinct targets are related to diseases, which may be treated or prevented by the use of antibodies, and wherein said composition is essentially free from immunoglobulin molecules that do not bind to one of said distinct targets.

2. The composition according to claim 1 wherein the constant region of said target-specific recombinant polyclonal antibodies belongs to the isotype of IgG, IgA, IgE, IgM, or IgD.

3. The composition according to claim 1, wherein the constant region(s) of said target-specific recombinant polyclonal antibodies belongs to one or two particular immunoglobulin subtype(s) of IgG or IgA.

4. The composition of claim 1, wherein the two or more sets of target-specific recombinant polyclonal antibodies have been expressed recombinantly in a single container.

5. The composition of claim 1, wherein at least one set of target-specific recombinant polyclonal antibodies comprises at least 3 distinct members.

6. The composition of claim 5, wherein each set comprises at least 3 distinct members.

7. The composition according to claim 1, wherein said diseases are selected from the group consisting of infectious diseases, allergy, and asthma and other respiratory diseases

8. The composition according to claim 1, wherein said distinct targets originate from a particular environmental niche.

9. The composition according to claim 8 wherein the environmental niche is selected from the group consisting of a hospital environment, a food-related environment, a water-related environment, a potential or actual site of biological warfare or bioterrorism, and/or a site with potential or actual exposure to pathogenic, toxic, potentially pathogenic, or potentially toxic agent(s).

10. The composition according to claim 8, wherein the distinct targets are related to pathogenic, toxic, potentially pathogenic or potentially toxic agents in hospitals, food or water or used in biological warfare or bioterrorism.

11. The composition according to claim 9, wherein said food-related environment includes the food as well as places where food is produced, tested or analyzed, or where food is served.

12. The composition according to claim 9, wherein said water-related environment includes drinking water, water used for food preparation, water used for other purposes where contact with an individual may cause health problems as well as any devices having been or which will come in contact with said water, as well as natural water reservoirs and streams.

13. The composition according to claim 9, wherein said site of biological warfare or bioterrorism includes geographical regions where there is an increased risk of biological warfare or bioterrorism or an actual incident has just occurred.

14. The composition according to claim 9, wherein said potential or actual site with pathogenic, toxic, potentially pathogenic or potentially toxic agents are selected from sites where there is an risk of pathogenic microorganisms, microbial toxins, poisonous plants or plant toxins, poisonous fungi or fungal toxins, poisonous and venomous animals or drug intoxication.

15. The composition according to claim 1, wherein said diseases are secondary infections associated with primary diseases.

16. The composition according to claim 15, wherein said secondary diseases are associated with other infections, cancer, transplantation, splenectomy, surgery, intravenous devices, assisted ventilation, multiple trauma or burns.

17. The composition according to claim 1, wherein the antibody members of a target-specific recombinant polyclonal antibody set mirrors the humoral immune response in a donor with respect to diversity, affinity and specificity against the distinct target.

18. The composition according to claim 1, wherein the antibody members of a target-specific recombinant polyclonal antibody set are encoded by nucleic acid sequences obtained from one or more human donors who have raised a humoral immune response against a distinct target and the polyclonal antibody is a fully human antibody.

19. The composition according to claim 1, wherein the antibody members of a set of target-specific recombinant polyclonal antibody are constituted of V_{H} and V_{L} pairs originally present in the donor(s).

20. A pharmaceutical composition comprising a composition according to claim 1 and one or more pharmaceutical acceptable carriers, excipients and/or diluents.

21. The pharmaceutical composition according to claim 20 further comprising at least one further therapeutic or prophylactic agent.

22. The pharmaceutical composition according to claim 21 wherein the pharmaceutical composition is directed against one or more secondary infections as well as the underlying primary disease.

23. The pharmaceutical composition according to 21, wherein the further therapeutic or prophylactic agent causes suppression of the immune system.

24. The pharmaceutical composition according to claim 20, further comprising one or more agents selected from the group consisting of antibiotics, chemotherapeutic agents, activity potentiators, hormones, cytokines, sedatives, analgesics and anti-inflammatory compounds.

25. A method for the manufacture of a composition comprising two or more sets of target-specific recombinant polyclonal antibodies, each set of target-specific recombinant polyclonal antibody capable of binding to a distinct target, wherein said distinct targets are related to diseases, which may be treated or prevented by the use of antibodies, and wherein said composition is essentially free from immunoglobulin molecules that do not bind to one of said distinct targets, said method comprising
(a) providing two or more cell lines wherein each cell line contains a number of different antibody clones capable of expressing unique antibodies,
(b) combining said cell lines in a single container and culturing the cells under conditions facilitating expression of said two or more sets of target-specific recombinant polyclonal antibodies; and
(c) recovering said two or more sets of target-specific recombinant polyclonal antibodies from the cell culture cells or cell culture supernatant.

26. The method according to claim 25, wherein said expression of each set of target-specific recombinant polyclonal antibodies takes place under conditions where the nucleic acid sequences encoding each antibody are integrated in the same site of the genome of each of the individual cells.

27. A polyclonal cell line comprising two or more parental polyclonal cell lines each expressing antibody molecules which are capable of binding to a distinct target.

28. A method for treatment or prophylaxis of an individual selected from the group consisting of
(a) an individual suffering from diseases selected from the group consisting of infectious diseases, allergy, and asthma and other respiratory diseases;
(b) an individual who has been or is in risk of being exposed to harmful or potentially harmful targets in a particular environmental niche;
(c) an individual suffering from one or more secondary infections, where said infections are associated with other infections, cancer, transplantation, splenectomy, surgery, intravenous devices, assisted ventilation, multiple trauma or burns;
(d) said method comprising administering to said individual a pharmaceutical composition according to claim 20.

29. The method of claim 28, wherein the individual is in a state of immunodeficiency or immunosuppression.

30. The method of claim 28, comprising a method for treatment or prophylaxis of one or more secondary infections as well as the underlying primary disease in an individual.

31. The method of claim 28, comprising additionally administering antibodies against T cells or other cells of the immune system to an individual having undergone transplantation surgery.

32. A kit for treatment or prophylaxis comprising a pharmaceutical composition according to claim 20 and in separate container(s) one or more further therapeutic agent.

33. A diagnostic kit for detecting the presence of targets originating from a particular environmental niche and relating to diseases which may be treated or prevented by the use of antibodies, said diagnostic kit comprising a composition according to claim 1.
